(19) United States
(12) Patent Application Publication
(10) Pub. No.: US 2009/0035824 A1
(43) Pub. Date: Feb. 5, 2009

(54) NUCLEIC ACID-TEMPLATED CHEMISTRY IN ORGANIC SOLVENTS

(76) Inventors: David R. Liu, Lexington, MA (US); Mary M. Rozenman, New York, NY (US); Christopher T. Calderone, St. Paul, MN (US)

Correspondence Address: GOODWIN PROCTER LLP;PATENT ADMINISTRATOR, 53 STATE STREET, EXCHANGE PLACE, BOSTON, MA, 02109-2881, US

(21) Appl. No.: 11/917,607

(22) PCT Filed: Jun. 16, 2006
(86) PCT No.: PCT/US06/23487

§ 371 Date: Sep. 12, 2008

Related U.S. Application Data
(60) Provisional application No. 60/691,409, filed on Jun. 17, 2005.

(51) Int. Cl. C12P 19/34; C07H 1/00; C40B 40/06; C07H 21/00; G01N 33/50

(52) U.S. Cl. 435/91.2; 536/25.3; 436/94; 506/16; 536/23.1

(57) ABSTRACT

The present invention provides methods and compositions for performing nucleic acid mediated chemistry in a variety of organic solvents. A variety of nucleic acid mediated chemical reactions may be efficiently carried out in organic solvents.

### RELATED APPLICATIONS

This application claims the benefit of and priority to U.S. Patent Application Ser. No. 60/691,409, filed Jun. 17, 2005, the entire disclosure of which is incorporated by reference herein for all purposes.

### GOVERNMENT FUNDING

The work described in this application was sponsored, in part, by NIH/NIGMS under Grant No. R01GM065865, and by the Office of Naval Research under Grant No. N00014-03-1-0749. The United States Government may have certain rights in the invention.

### FIELD OF THE INVENTION

The invention relates generally to nucleic acid-mediated chemistry. More particularly, the invention relates to nucleic acid-mediated chemistry conducted in solutions having one or more organic solvents.

### BACKGROUND OF THE INVENTION

Nucleic acid-templated synthesis (DNA-templated synthesis or DTS) enables new modes of controlling chemical reactivity and allows evolutionary principles to be applied to the discovery of synthetic small molecules, synthetic polymers, and new chemical reactions. Li et al. (2004) ANGEW. CHEM. INT. ED. 43: 4848-4870; Calderone et al. (2002) ANGEW. CHEM. INT. ED. 41: 4104-4108; Sakurai et al. (2005) J. AM. CHEM. SOC. 127: 1660-166; Gartner et al. (2004) SCIENCE 305: 1601-1605; Rosenbaum et al. (2003) J. AM. CHEM. SOC. 125: 13924-13925; Kanan et al. (2004) NATURE 431: 545-549.

While there have been attempts to use quaternary ammonium salts that can associate with DNA phosphates to form complexes that are soluble in organic solvents, see, Ijiro et al. (1992) J. CHEM. SOC. CHEM. COMM. 18: 1339 1341; Tanaka et al. (1996) J. AM. CHEM. SOC. 118:10679-10683; Bromberg et al. (1994) PROC. NATL. ACAD. SCI. U.S.A. 91, 143-147; Mel'nikov et al. (1999) LANGMUIR 15: 1923-1928; Mel'nikov et al. (1995) J. AM. CHEM. SOC. 117, 2401-2408; Mel'nikov et al. (1995) J. AM. CHEM. SOC. 117: 9951-9956; Sergeyev et al. (1999) J. AM. CHEM. SOC. 121: 1780-1785; Kabanov et al. (1995) MACROMOLECULES 28: 3657-3663; Sergeyev et al. (1999) LANGMUIR 15: 4434-4440, there remains a need for simple, efficient and sequence-specific methodologies that permit nucleic acid-templated reactions to be performed in various organic solvents. There also remains a need for access to reagents that are insoluble in water as well as reactions in which the participation of water precludes product formation.

### SUMMARY

The present invention is based, in part, upon the discovery of simple, efficient and sequence-specific methods to perform DNA-templated synthesis in a variety of organic solvents. DNA-templated synthesis enables biological principles to be applied to the creation and discovery of synthetic molecules. Ions (e.g., micromolar concentrations of tetraalkylammonium salts) can be used to render DNA duplexes soluble in a variety of organic solvents. These methods enable reactions that are inaccessible in water, e.g., Pd₂(dba)-3-mediated Heck coupling or pyrrolidine-catalyzed aldol condensation, to be performed in a DNA-templated format. In addition, conditions needed to perform DTS in organic solvents also support reactions that are known to take place in aqueous solution, e.g., amine acylation, Wittig olefination, and Pd(II)-mediated alkyne-alkene coupling.

The methods of the present invention increase the structural diversity that can be accessed through DTS by enabling the use of intermediates or non-DNA-linked reactants that are insoluble or unstable in aqueous solvents. The invention provides DNA duplexes that are sufficiently soluble and stable in organic solvents under the conditions that support DNA template-directed synthesis. These discoveries allow the significant expansion the scope of DTS-based approaches to the discovery of small-molecules, synthetic polymers, and new chemical reactions.

In one aspect, the invention provides a method of performing nucleic acid templated synthesis to produce a reaction product. The method includes providing a solution which includes a template and a transfer unit. The template includes a first reactive unit that is associated with a first oligonucleotide defining a first codon sequence. The transfer unit includes a second reactive unit associated with a second oligonucleotide that defines a first anti-codon sequence complementary to the first codon sequence of the template. The first codon and first anti-codon sequences are annealed to bring the first reactive unit and the second reactive unit into reactive proximity. Thereafter, a reaction between the first and second reactive units is induced in a solution that includes an organic solvent to produce a reaction product. In one embodiment, the above method further includes the additional step of adding a solution containing an organic solvent to the product of the annealing step.

In another aspect, the invention provides a method of performing nucleic acid templated synthesis to produce a reaction product. The method includes providing a solution that includes an organic solvent, a template and a transfer unit. The template includes a first reactive unit associated with a first oligonucleotide that defines a first codon sequence. The transfer unit includes a second reactive unit associated with a second oligonucleotide that defines a first anti-codon sequence complementary to the first codon sequence of the template. The first codon and first anti-codon sequences are annealed to bring the first reactive unit and the second reactive unit into reactive proximity. A reaction between the first and second reactive units is induced to produce the reaction product. In one embodiment, all of the above steps are performed in a single solution that includes an organic solvent.

In one embodiment of the template, the first reactive unit is associated with the first oligonucleotide at a location adjacent to an end of the first oligonucleotide. In another embodiment of the template, the first reactive unit is associated with the first oligonucleotide at a location at least 2 bases from an end of the first oligonucleotide. In a more detailed embodiment, the first reactive unit is associated with the first oligonucleotide at a location at least 3, 4, 5, 6, 7, 8, 9 or 10 bases from an end of the first oligonucleotide. In another embodiment, the template is capable of producing an omega or single stranded loop structure when annealed to the transfer unit.

To facilitate a nucleic acid-templated reaction, the template and/or the transfer units may be solubilized by one or more quaternary ammonium ions. Exemplary ions include ions of the formula ⁺NR₁R₂R₃R₄. Each of the R's may be the same or different unsubstituted or substituted alkyl groups, e.g., alkyl groups with 1, 2, 3, 4, 5, 6, 7, 8, 9, 10, 11, 12, 13, 14, 15, 16, 17, 18, 19, or more carbon atoms. The alkyl groups may be branched with one, two three or more substitutions.

In yet another aspect, the invention provides a method for identifying a compound having binding affinity to a target molecule. The method includes performing one or more nucleic acid-templated reactions to produce one or more compounds each of which is covalently linked to a corresponding oligonucleotide having a nucleotide sequence informative of the synthetic history or structure of the compound. At least one of the nucleic acid-templated reactions is performed in a solution that includes an organic solvent. The compounds produced are mixed with a target molecule under conditions to permit the compounds capable of binding the target molecule to bind thereto. The compounds that bind to the target molecule are separated from unbound compounds. The oligonucleotide associated with a compound that binds to the target molecule is identified as indicative of binding affinity of the compound to the target molecule. In one embodiment, the nucleotide sequence associated with a particular compound encodes the synthesis of that compound.

In addition, the invention provides reaction products and libraries of compounds prepared by any of the foregoing methods.

The foregoing aspects and embodiments of the invention may be more fully understood by reference to the following figures, detailed description and claims.

### DEFINITIONS

The term, “associated with” as used herein describes the interaction between or among two or more groups, moieties, compounds, monomers, etc. When two or more entities are “associated with” one another as described herein, they are linked by a direct or indirect covalent or non-covalent interaction. Preferably, the association is covalent. The covalent association may be, for example, but without limitation, through an amide, ester, carbon-carbon, disulfide, carbamate, ether, thioether, urea, amine, or carbonate linkage. The covalent association may also include a linker moiety, for example, a photocleavable linker. Desirable non-covalent interactions include hydrogen bonding, van der Waals interactions, dipole-dipole interactions, pi stacking interactions, hydrophobic interactions, magnetic interactions, electrostatic interactions, etc. Also, two or more entities or agents may be “associated with” one another by being present together in the same composition.

The term, “biological macromolecule” as used herein refers to a polynucleotide (e.g., RNA, DNA, RNA/DNA hybrid), protein, peptide, lipid, or polysaccharide. The biological macromolecule may be naturally occurring or non-naturally occurring. In a preferred embodiment, a biological macromolecule has a molecular weight greater than about 5,000 Daltons.

The terms, “polynucleotide,” “nucleic acid”, or “oligonucleotide” as used herein refer to a polymer of nucleotides, at least three nucleotides in length. The polymer may include, without limitation, natural nucleosides (i.e., adenosine, thymidine, guanosine, cytidine, uridine, deoxyadenosine, deoxythymidine, deoxyguanosine, and deoxycytidine), nucleoside analogs (e.g., 2-aminoadenosine, 2-thiothymidine, inosine, pyrrolo-pyrimidine, 3-methyl adenosine, 5-methylcytidine, C5-bromouridine, C5-fluorouridine, C5-iodouridine, C5-propynyl-uridine, C5-propynyl-cytidine, C5-methylcytidine, 7-deazaadenosine, 7-deazaguanosine, 8-oxoadenosine, 8-oxoguanosine, O(6)-methylguanine, and 2-thiocytidine), chemically modified bases, biologically modified bases (e.g., methylated bases), intercalated bases, modified sugars (e.g., 2′-fluororibose, ribose, 2′-deoxyribose, arabinose, and hexose), or modified phosphate groups (e.g., phosphorothioates and 5′-N-phosphoramidite linkages). Nucleic acids and oligonucleotides may also include other polymers of bases having a modified backbone, such as a locked nucleic acid (LNA), a peptide nucleic acid (PNA), a threose nucleic acid (TNA) and any other polymers capable of serving as a template for an amplification reaction using an amplification technique, for example, a polymerase chain reaction, a ligase chain reaction, or non-enzymatic template-directed replication.

The term, “small molecule” as used herein, refers to an organic compound either synthesized in the laboratory or found in nature having a molecular weight less than 10,000 grams per mole, optionally less than 5,000 grams per mole, and optionally less than 2,000 grams per mole.

The terms, “small molecule scaffold” or “molecular scaffold” as used herein, refer to a chemical compound having at least one site or chemical moiety suitable for functionalization. The small molecule scaffold or molecular scaffold may have two, three, four, five or more sites or chemical moieties suitable for functionalization. These functionalization sites may be protected or masked as would be appreciated by one of skill in this art. The sites may also be found on an underlying ring structure or backbone.

The term, “transfer unit” as used herein, refers to a molecule comprising an oligonucleotide having an anti-codon sequence associated with a reactive unit including, for example, but not limited to, a building block, monomer, monomer unit, molecular scaffold, or other reactant useful in template mediated chemical synthesis.

The term, “template” as used herein, refers to a molecule comprising an oligonucleotide having at least one codon sequence suitable for a template mediated chemical synthesis. The template optionally may comprise (i) a plurality of codon sequences, (ii) an amplification means, for example, a PCR primer binding site or a sequence complementary thereto, (iii) a reactive unit associated therewith, (iv) a combination of (i) and (ii), (v) a combination of (i) and (iii), (vi) a combination of (ii) and (iii), or a combination of (i), (ii) and (iii).

The terms, “codon” and “anti-codon” as used herein, refer to complementary oligonucleotide sequences in the template and in the transfer unit, respectively, that permit the transfer unit to anneal to the template during template mediated chemical synthesis.

### BRIEF DESCRIPTION OF THE DRAWINGS

The invention may be further understood from the following figures in which:

FIG. 1A is a schematic representation of an exemplary embodiment of a method of performing DNA-templated synthesis in organic solvents. FIG. 1B is a schematic representation of exemplary DNA template architectures for performing DNA-templated synthesis in organic solvents. FIG. 1C shows denaturing polyacrylamide gel electrophoresis (PAGE) analysis of aqueous amine acylation (40 mM EDC and 25 mM sNHS, lanes 1-4) performed in aqueous 3-(N-morpholino)propane sulfonic acid (MOPS; 100 mM, pH 7.0 with 1 M NaCl) for 12 h at 25° C. The same reaction (with NHS instead of sNHS) was carried out in 95% MeCN for 12 h at 25° C. with (lanes 5-8) or without (lanes 9-12) oligonucleotide pre-hybridization. Reactions with mismatched oligonucleotides (mis) are shown in lanes 4, 8, and 12.

FIG. 2A shows denaturing PAGE analysis of aqueous reductive amination (40 mM NaBH₃CN, lanes 1-4), Wittig olefination (lanes 5-8), and Pd^{II}-mediated alkene-alkyne coupling (1 mM Na₂PdCl₄)(lanes 9-12) reactions. All aqueous reactions were performed in MOPS (100 mM, pH 7.0 with 1 M NaCl). The reductive amination and Wittig olefination reactions were conducted for 12 hours at 25° C., while the Pd^{II} coupling was conducted for 4 hours at 37° C. FIG. 2B shows denaturing PAGE analysis of the reactions described for FIG. 2A, but performed in 95% DMF (amination) or 95% MeCN (olefination and coupling) with 5% aqueous solvent for 12 hours at 25° C. (amination and olefination) or for 4 hours at 37° C. (coupling). The Wittig olefination reaction in 95% MeCN contained 10 mM NaOH. Reagent types are labeled as in FIG. 1C.

FIG. 3 shows DNA-templated reactions enabled by organic solvents as analyzed by denaturing PAGE. The left PAGE analysis shows the outcome of a secondary amine-catalyzed aldol condensation in 95% MeCN, with 50 mM pyrrolidine (lanes 1-4). The right PAGE analysis shows the outcome of a Pd₂(dba)-3-mediated Heck coupling in 95% THF (lanes 5-8). All reactions were carried out for 16 hours at 25° C. Reagent types are labeled as in FIG. 1C.

FIG. 4 shows denaturing PAGE analysis of DNA-templated reactions carried out in dry organic solvents (i.e., >99.9% organic solvent). The left PAGE analysis shows the outcome of a Wittig olefination (100 mM TEA in >99.9% MeCN, lanes 1-4). The center PAGE analysis shows the outcome of a secondary amine-catalyzed aldol condensation (50 mM pyrrolidine in >99.9% MeCN, lanes 5-8). The right PAGE analysis shows the outcome of an amine acylation (40 mM DCC and 25 mM NHS in >99.9% DCM, lanes 9-12). All oligonucleotide pairs were pre-hybridized, frozen, and lyophilized to dryness before the addition of the anhydrous solvents and reagents listed above. Reactions were performed at 25° C. for 12 hours (acylation and olefination) or 16 hours (aldol). Reagent types are labeled as in FIG. 1C.

FIG. 5 shows denaturing PAGE analysis of enamine aldol chemistry (lanes 1-4) and Heck coupling chemistry (lanes 5-8) in aqueous solvent.

FIG. 6 shows denaturing PAGE analysis of DTS chemistry in 99% organic and 1% aqueous solvent. FIG. 6A shows the results of amine acylation reactions (lanes 1-4), Wittig olefinations (lanes 5-8), and Pd(II) coupling reactions (lanes 9-12). FIG. 6B shows the result of reductive amination reactions (lanes 1-4), aldol condensation reactions (lanes 5-8), and Heck coupling reactions (lanes 9-12).

FIG. 7 shows denaturing PAGE analysis of water titration in the aldol reaction, for E1 and Ω5 architectures.

FIG. 8 is a schematic representation of an exemplary embodiment of a method for performing DNA-templated synthesis in organic solvents in the presence of alkyl ammonium salts.

FIG. 9 shows denaturing PAGE analysis of representative DNA-templated chemistries in organic solvents in the presence of alkylammonium salts. FIG. 9A shows results of Heck coupling reactions (lanes 1-4) and aldol condensation reactions (lanes 5-8), both in the presence of cetyltrimethylammonium bromide (CTAB). FIG. 9B shows the results of amine acylation reactions (lanes 1-4), Wittig olefination reactions (lanes 5-8), and Pd(II) coupling reactions (lanes 9-12), all in the presence of CTAB.

FIG. 10 is a schematic representation of an exemplary scheme for MALDI analysis of DNA-templated reaction products.

### DESCRIPTION OF THE INVENTION

The present invention provides simple, efficient and sequence-specific methods to perform DNA-templated synthesis in organic solvents (e.g., acetonitrile, DMF and THF), with low (for example, less than 10%) or minimal water content. These methods increase the structural diversity that can be accessed through DTS by enabling the use of intermediates or non-DNA-linked reactants that are insoluble or unstable in aqueous solvents. The invention provides DNA duplexes that are sufficiently soluble and stable in organic solvents under the conditions that support DNA template-directed synthesis. These discoveries allow the significant expansion the scope of DTS-based approaches to the discovery of small-molecules, synthetic polymers, and new chemical reactions.

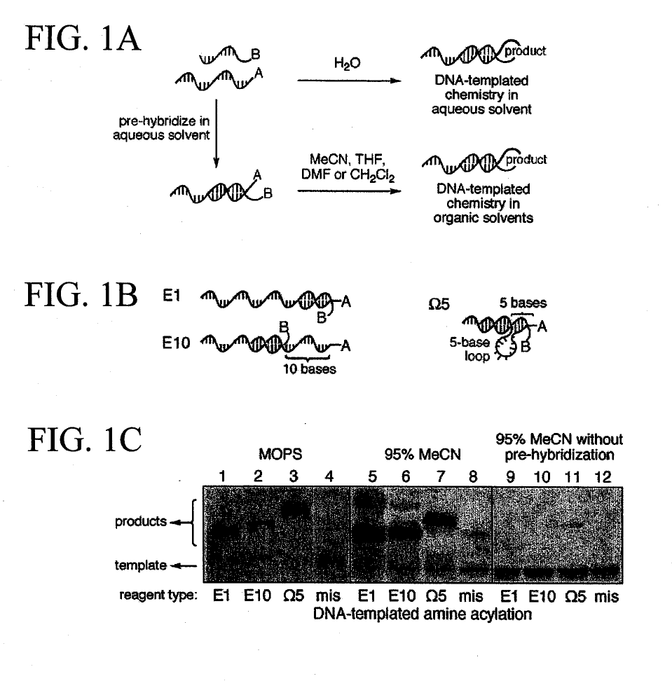

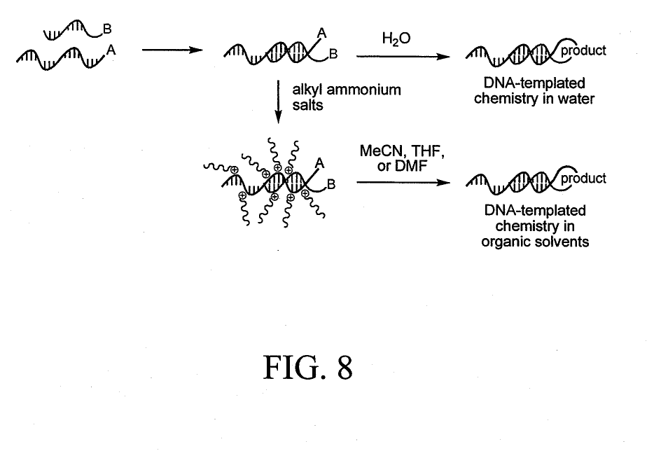

Among other things, it has found that a short (10-30 bp) DNA duplex formed in aqueous solution and then transferred to an organic solvent containing low concentrations (μM) of quaternary ammonium salts retained its double-stranded structure (FIG. 8). Indeed, DNA-templated chemistry was found to take place efficiently and sequence-specifically in organic solvents in the presence of alkyl ammonium salts (see Example). It is contemplated that at the extremely low concentrations required for DTS (nM), alkyl ammonium salts might not be necessary for the solubilization of duplexes preformed in aqueous solution (FIG. 1A).

To evaluate the ability of preformed duplexes to support DTS in primarily organic solvents, three known DNA-templated chemistries in four distinct contexts were first investigated (FIG. 1B): (i) in a simple end-of-helix architecture with juxtaposed reactants (E1), (ii) in a long-distance end-of-helix architecture with 10 intervening nucleotides between hybridized reactants (E10), (iii) in the “omega” architecture (Gartner et al. (2003) ANGEW. CHEM. INT. ED. 42: 1370-1375) with a 5-base loop (Ω5), and (iv) with reactants linked to non-complementary (mismatched) oligonucleotides. Products were characterized both by denaturing PAGE analysis and by MALDI mass spectrometry (see Table 1).

TABLE 1
MALDI-TOF analysis of DNA-templated reactions in organic solvents

| Template | Reagent | Product | Product mass | Solvent |
|---|---|---|---|---|
| 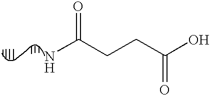 | 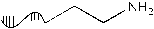 | 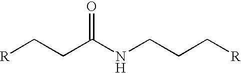 | 3429 ± 5(3430.7) | 95% MeCN |
| 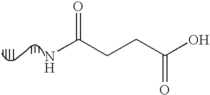 | 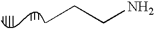 | 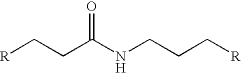 | 3433 ± 5(3430.7) | 99.9% DCM |
| 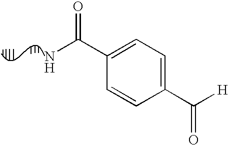 | 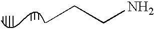 | 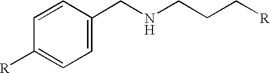 | 3463 ± 5(3464.7) | 95% DMF |
| 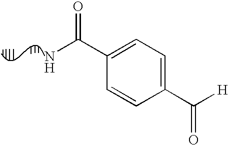 | 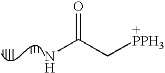 | 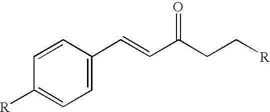 | 3505 ± 5(3504.7) | 95% MeCN or95% DMF |
| 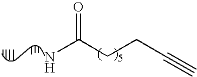 | 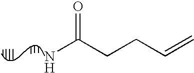 | 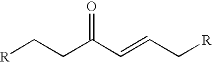 | 3584 ± 5(3582.8) | 95% MeCN |
| 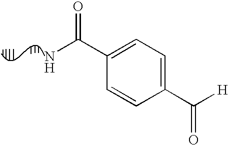 | 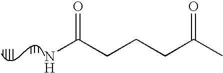 | 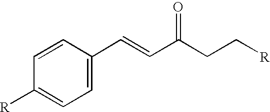 | 3579 ± 5(3574.8) | 95% MeCN |
| 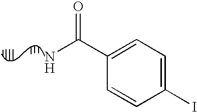 | 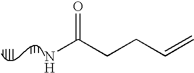 | 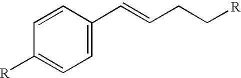 | 3536 ± 5(3532.8) | 99% THF |

DNA-templated amine acylation mediated by 1-(3-dimethylaminopropyl)-3-ethyl carbodiimide (EDC) and N-hydroxysulfosuccinimide (SNHS) has been well characterized in aqueous solution (Gartner et al. (2003) ANGEW. CHEM. INT. ED. 42: 1370-1375) and is known to take place efficiently even when reactive groups are separated by many intervening nucleotides. To carry out DNA-templated amine acylation in organic solvent, template and reagent oligonucleotides (Table 1) were pre-hybridized in a small volume of aqueous 70 mM NaCl. Amine acylation was initiated by the addition of organic solvent containing 40 mM EDC and 25 mM N-hydrosuccinimide (NHS) to result in a final solvent composition of 95% acetonitrile and 5% water. Under these conditions, the E1, E1, and Ω5 architectures all generated amide products efficiently (88, 82, and 70% yield, respectively), as characterized by denaturing PAGE and MALDI mass spectrometry (FIG. 1C, lanes 5-8; Table 1). When the same reaction was carried out in a final solvent composition of 95% acetonitrile with 5% water and 50 μM cetyltrimethylammonium bromide (CTAB) the E1, E10, and Ω5 architectures generated amide products in 58, 63, and 82% yield, respectively (see Example, below). These results demonstrate that alkylammonium salts are not needed in order to perform DNA-templated chemistry in nonaqueous solvents with low water content (i.e., less than or equal to 5% water).

For comparison, in aqueous solution (100 mM MOPS, pH 7.0, 1 M NaCl, 40 mM EDC, 25 mM sNHS) the identical amine acylation reactions proceeded in 81, 58, and 84% yield, respectively (FIG. 1C, lanes 1-4). Importantly, significant product formation when the reagent and template oligonucleotides were mismatched in either solvent was not observed; indicating that DTS in 95% acetonitrile retains sequence specificity (FIG. 1C). Moreover, no product formation was observed when the template and reagent oligonucleotides were not pre-hybridized but instead were added separately to organic solvent containing 40 mM EDC and 25 mM NHS; suggesting that duplexes should be preformed before exposure to organic solvent in order to generate products. (FIG. 1C, lanes 9-12).

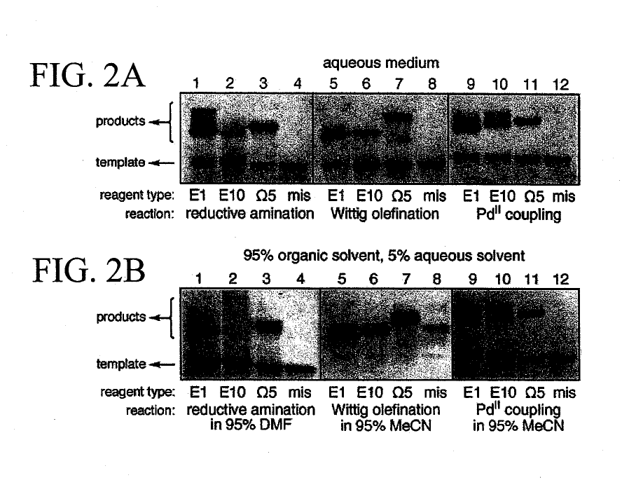

Next, three diverse DNA-templated reactions in organic and aqueous solvents were compared. Reductive amination of an aldehyde, Wittig olefination (Gartner et al. (2002) J. AM. CHEM. SOC. 124: 10304-10306) between aryl aldehydes and phosphoranes, and Pd^{II}-mediated alkene-alkyne coupling to generate an enone (Kanan et al. (2004) NATURE 431: 545-549) all exhibited comparable reactivity patterns in aqueous solution or in 95% organic solvent in all four contexts examined (FIG. 2, Table 1).

In 95% N,N′-dimethylformamide (DMF), the E1, E10, and Ω5 architectures generated reductive amination products in 59, 5, and 45% yield, respectively (FIG. 2B, lanes 1-4). The distance dependence (E1 vs. E10 reactivity difference) observed in DNA-templated reductive amination reactions was similar to that seen in the aqueous system (FIG. 2A) and was consistent with previous findings (Gartner et al. (2003) ANGEW. CHEM. 115: 1408-1413, (2003) ANGEW. CHEM. INT. ED. 42: 1370-1375). Wittig chemistry proceeded efficiently in 95% acetonitrile with product yields exceeding 90% in the E1, E10, and Ω5 architectures (FIG. 2B, lanes 5-8). Similarly, in 95% acetonitrile, enone products were generated in 71, 60, and 63% yield in the E1, E10, and Ω5 architectures, respectively (FIG. 2B, lanes 9-11). No significant product formation was observed when oligonucleotide sequences were mismatched (FIG. 2B). These results indicate that DNA-templated carbon-carbon bond formation can be carried out efficiently and sequence-specifically in wet organic solvents (i.e., less than or equal to 99.9% organic solvent) even over ten intervening template nucleotides.

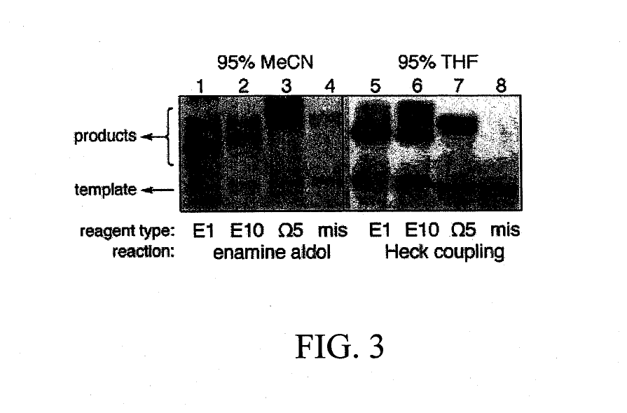

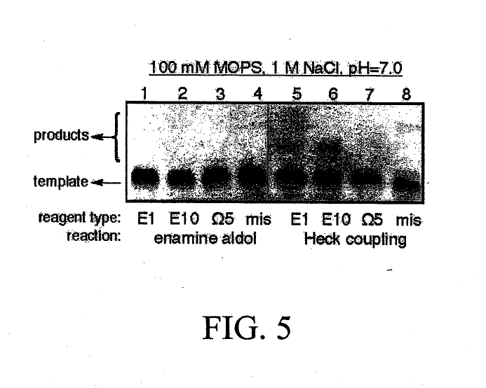

Reactions that are inaccessible in water may also be used in nucleic acid-templated chemistry. Although DNA-linked small molecules are generally soluble in water, many small-molecule reagents and catalysts are not. For example, the DNA-templated Pd₂(dba)-3-mediated Heck coupling (Beletskaya et al. (2000) CHEM. REV. 100: 3009-3066) of an unactivated alkene and an aryl iodide generated no observable products in aqueous solution (FIG. 5, lanes 5-8), presumably because the Pd₂(dba)₃ complex is not water-soluble. In contrast, this Heck coupling proceeded sequence-specifically in yields of 91%, 85%, and 80% in the E1, E10, and Ω5 architectures, respectively, in 95% tetrahydrofuran (THF) (FIG. 3, Table 1). Significant product formation was also observed when reactions were carried out in 99% THF (see Example, below).

A second class of reactions that are inaccessible in aqueous solutions are those that proceed through a water-incompatible mechanism. Although synthesis involving imine intermediates has been performed in various aqueous systems, (Koh et al. (1994) J. AM. CHEM. SOC. 116: 11234-11240; Wagner et al. (1995) SCIENCE 270: 1797-1800; Wei et al. (2002) J. AM. CHEM. SOC. 124: 5638-5639) reactions making use of unstabilized imines are predominantly carried out in organic solvents to avoid the tendency of imines to undergo hydrolysis. (Cordes et al. (1963) J. AM. CHEM. SOC. 85: 2843-2848). In these experiments, pyrrolidine-catalyzed aldol reactions were compared (Stork et al. (1954) J. AM. CHEM. SOC. 76: 2029-2030) between an aldehyde-linked template and a ketone-linked reagent oligonucleotide in aqueous or organic solvents. In 95% acetonitrile, the E1, E10, and Ω5 architectures generated aldol condensation products in 88%, 79%, and 82% yield, respectively (FIG. 3, lanes 1-4, Table 1). In contrast, no significant product formation was observed when these reactions were carried out in aqueous solutions (FIG. 5).

Aldol product formation was distance-independent (Gartner et al. (2002) J. AM. CHEM. SOC. 124: 10304-10306) and no product formation was observed with sequence-mismatched reactants (FIG. 3). Without wishing to be bound by theory, it is contemplated that in the aqueous system, imine hydrolysis competes effectively with tautomerization to the nucleophilic enamine species. (No DNA-Templated enamine aldol reactivity was observed at concentrations of H₂O greater than 25% in CH₃CN. See Example (FIG. 5). Additionally, reversible aldol addition is more likely in water than the dehydrative aldol condensation observed in acetonitrile. The ability of DTS in organic solvents to support pyrrolidine-catalyzed aldol reactions is especially significant in light of recent advances in asymmetric enamine-based organocatalytic transformations. (List (2004) ACC. CHEM. RES. 37: 548-557; Northup et al. (2004) SCIENCE 305: 1753-1755).

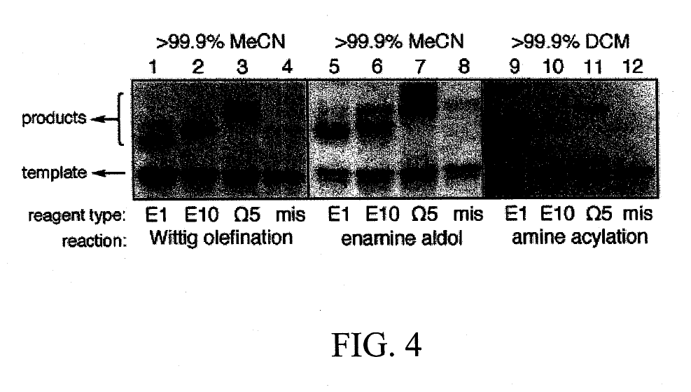

To test the possibility of performing DTS in anhydrous organic solvents, DNA-templated aldol, Wittig olefination, and amine acylation reactions in dry solvents (e.g., >99.9% organic solvent) were tested. Pre-hybridized DNA-linked reactants were lyophilized to dryness and then dissolved in anhydrous organic solvents. This treatment resulted in an organic solvent content of >99.9% with the final water content of the reaction measured to be 300-600 ppm by Karl Fischer analysis. Sequence-specific aldol and Wittig product formation under these conditions was observed, albeit at lower yields (10-56%) than in 95% organic solvents (FIG. 4, lanes 1-8). Amine acylation in >99.9% dichloromethane (DCM) containing 40 mM of water-insoluble dicyclohexyl carbodiimide (DCC) and 25 mM NHS proceeded only in low efficiencies (21% for the E1 architecture and <10% yield for the E10 and Ω5 architectures, FIG. 4, lanes 9-12, Table 1), although sequence-specificity was retained. Without wishing to be bound by theory, it is contemplated that a minimal level of hydration around the DNA backbone may significantly enhance DNA-templated reactions, probably by stabilizing the template-reagent duplex.

In one aspect, the invention provides a method of performing nucleic acid templated synthesis to produce a reaction product. The method includes providing a solution that includes a template and a transfer unit. The template includes a first reactive unit that is associated with a first oligonucleotide defining a first codon sequence. The transfer unit includes a second reactive unit associated with a second oligonucleotide that defines a first anti-codon sequence complementary to the first codon sequence of the template. The first codon and first anti-codon sequences are annealed to bring the first reactive unit and the second reactive unit into reactive proximity. Thereafter, a reaction between the first and second reactive units is induced in a solution that includes an organic solvent to produce a reaction product.

In one embodiment, the above method further includes the additional step of adding a solution containing an organic solvent to the product of the annealing step.

In another aspect, the invention provides a method of performing nucleic acid templated synthesis to produce a reaction product. The method includes providing a solution that includes an organic solvent, a template and a transfer unit. The template includes a first reactive unit associated with a first oligonucleotide that defines a first codon sequence. The transfer unit includes a second reactive unit associated with a second oligonucleotide that defines a first anti-codon sequence complementary to the first codon sequence of the template. The first codon and first anti-codon sequences are annealed to bring the first reactive unit and the second reactive unit into reactive proximity. A reaction between the first and second reactive units is induced to produce the reaction product. In one embodiment, all of the steps above are performed in a single solution that includes an organic solvent.

In one embodiment of the template, the first reactive unit is associated with the first oligonucleotide at a location adjacent to an end of the first oligonucleotide. In another embodiment of the template, the first reactive unit is associated with the first oligonucleotide at a location at least 2 bases from an end of the first oligonucleotide. In a more detailed embodiment, the first reactive unit is associated with the first oligonucleotide at a location at least 3, 4, 5, 6, 7, 8, 9 or 10 bases from an end of the first oligonucleotide.

In another embodiment, the template is capable of producing an omega or a single stranded loop structure when annealed to the transfer unit.

The first reactive unit may be covalently attached to the first oligonucleotide. Additionally, the second reactive unit may be covalently attached to the second oligonucleotide. In one embodiment, the method further includes performing one or more chemical reactions not mediated by nucleic acid templates. In another embodiment, the method includes one or more chemical reactions that involve reactants not associated with oligonucleotides. In another embodiment, the method includes one or more chemical reactions that involve reactants not covalently linked to oligonucleotides.

Any organic solvent that facilitates nucleic acid-templated chemistry may be used in the present invention. Exemplary solvents include CH₃CN, DMF, THF, CH₃OH, CH₂Cl₂, CCl₄, CHCl₃, toluene, benzene, diethyl ether, glyme, hexanes, and DMSO. The weight percentage of organic solvent in a solution may be 100% or the weight percentage may be 99%, 98%, 97%, 95%, 90%, 80%, 70%, 60%, 50% organic solvent or more or less than any of the foregoing percentages. The weight percentage of water in a solution may be 0% or may be 1%, 2%, 3%, 5%, 10%, 20%, 30%, 40% water, or more or less than any of the foregoing percentages.

The template may further include a second, different codon sequence. A second transfer unit may be employed that anneals to the second, different codon sequence of the template. Additionally, the template may include a third, fourth or more codon sequences. A third, fourth or more transfer units may be employed that anneal to the third, fourth or additional codon sequence of the template, respectively. The first, second (third, fourth or more) transfer units may be provided individually or together in an effort to control the reactions and the formation of reaction products. The reaction product may be covalently attached to the template. The method of the invention may further include an additional step of selecting reaction products (e.g., associated with the template) which may include amplifying the template and/or determining the sequence of the template thereby to facilitate identification of the reaction product.

The nucleic acid templated reactions that may be utilized by the present invention may or may not be performed in an aqueous medium, both water compatible and water-incompatible reactions are useful in the present invention. The nucleic acid-templated reaction in organic solvent may produce a smaller or greater yield of product than the reaction in an aqueous solvent. In one embodiment, the nucleic acid templated reaction is water-incompatible and could not otherwise performed in aqueous medium.

Any nucleic acid-templated reaction that may be performed in a solution having an organic solvent may be utilized in the present invention. In one embodiment, the nucleic acid-templated reaction is a carbon-carbon bond formation reaction. Exemplary nucleic acid-templated reactions include reactions catalyzed by organometallic catalysts, asymmetric reactions, Wittig reactions, Witting-type reactions, Pd coupling reactions, Heck coupling, aldol, pyrrolidine-catalyzed aldol reactions, acylations, and amine acylations. To facilitate a nucleic acid-templated reaction, the template and/or the transfer units may be solublized by one or more quaternary ammonium ions. Exemplary ions include ions of the formula ⁺NR₁R₂R₃R₄. Each of the R's may be the same or different unsubstituted or substituted alkyl groups, e.g., alkyl groups with 1, 2, 3, 4, 5, 6, 7, 8, 9, 10, 11, 12, 13, 14, 15, 16, 17, 18, 19, or more carbon atoms. The alkyl groups may be branched with one, two three or more substitutions.

In certain embodiments, the yield of product may be enhanced when the template and transfer units are pre-annealed in an aqueous solvent. For example, the yield achieved by prehybridization in aqueous solvent may be greater by 30%, 40%, 60%, 80%, or 90% than the yield achieved when there is no prehybridization in aqueous solvent. Furthermore, it is contemplated that in certain embodiments, the product yield achieved by reactions performed in an aqueous/organic solvent mixture can be greater than reactions performed (i) in aqueous solvent alone, (ii) in dry organic solvent, or (iii) in both aqueous solvent alone and in dry organic solvent.

In yet another aspect, the invention provides a method for identifying a compound having binding affinity to a target molecule. The method includes performing one or more nucleic acid-templated reactions to produce one or more compounds each of which is covalently linked to a corresponding oligonucleotide having a nucleotide sequence informative of the synthetic history or structure of the compound. At least one of the nucleic acid-templated reactions is performed in a solution that includes an organic solvent. The compounds produced are mixed with a target molecule under conditions to permit the compounds capable of binding the target molecule to bind thereto. The compounds that bind to the target molecule are separated from unbound compounds. The oligonucleotide associated with a compound that binds to the target molecule is identified as indicative of binding affinity of the compound to the target molecule. In one embodiment, the nucleotide sequence associated with a particular compound encodes the synthesis of that compound.

The target molecule may be any compound of interest, small molecule or polymeric, naturally occurring or non-naturally occurring, and biological molecules or otherwise. A target can be an enzyme, protein, peptide, carbohydrate, polysaccharide, glycoprotein, hormone, receptor, antigen, antibody, virus, substrate, metabolite, transition state analog, cofactor, inhibitor, drug, dye, nutrient, growth factor, cell, tissue etc., without limitation. For example, the binding region of a target molecule may include a catalytic site of an enzyme, a binding pocket on a receptor (e.g., a G-protein coupled receptor), a protein surface area involved in a protein-protein or protein-nucleic acid interaction (e.g., a hot-spot region), or a specific site on DNA (e.g., the major groove). The natural function of the target could be stimulated (agonized), reduced (antagonized), unaffected, or completely changed by the binding depending on the precise binding mode and the particular binding site. A target can also be a surface of a material, e.g., the surface or coating of a polymeric material or a metallic material.

For example, a target and a small molecule having binding affinity toward the target may form a non-covalently interaction to associate the target with the binding molecule. Non-covalent binding includes the subsequent introduction of functional groups into the small molecule compound that causes covalent attachment to the target following the non-covalent molecular recognition and binding event.

Examples of targets include kinases, phosphatases, proteases, receptors, ion channels, oxidases and reductases, catabolic and anabolic enzymes, pumps, and electron transport proteins.

In addition, the invention provides reaction products and libraries of compounds prepared by any of the foregoing methods.

Various aspects of nucleic acid-templated chemistry are discussed in detail below. Additional information may be found in U.S. Patent Application Publication Nos. 2004/0180412 A1 (U.S. Ser. No. 10/643,752) by Liu et al. and 2003/0113738 A1 (U.S. Ser. No. 10/101,030) by Liu et al., and in U.S. Patent Application Ser. No. 60/661,039 by Askenazi et al.

### I. Template Considerations

The nucleic acid template can direct a wide variety of chemical reactions without obvious structural requirements by sequence-specifically recruiting reactants linked to complementary oligonucleotides. As discussed, the nucleic acid-mediated format permits reactions that may not be possible using conventional synthetic approaches. During synthesis, the template hybridizes or anneals to one or more transfer units to direct the synthesis of a reaction product, which during certain steps of templated synthesis remain associated with the template. A reaction product then is selected or screened based on certain criteria, such as the ability to bind to a preselected target molecule. Once the reaction product has been identified, the associated template can then be sequenced to decode the synthetic history of the reaction product. Furthermore, as will be discussed in more detail below, the template may be evolved to guide the synthesis of another chemical compound or library of chemical compounds.

### (i) Template Format

The template may incorporate a hairpin loop on one end terminating in a reactive unit that can interact with one or more reactive units associated with transfer units. For example, a DNA template can comprise a hairpin loop terminating in a 5′-amino group, which may or may not be protected. The amino group may act as an initiation point for formation of an unnatural polymer or small molecule.

The length of the template may vary greatly depending upon the type of the nucleic acid-templated synthesis contemplated. For example, in certain embodiments, the template may be from 10 to 10,000 nucleotides in length, from 20 to 1,000 nucleotides in length, from 20 to 400 nucleotides in length, from 40 to 1,000 nucleotides in length, or from 40 to 400 nucleotides in length. The length of the template will of course depend on, for example, the length of the codons, the complexity of the library, the complexity and/or size of a reaction product, the use of spacer sequences, etc.

### (ii) Codon Usage

It is contemplated that the sequence of the template may be designed in a number of ways without going beyond the scope of the present invention. For example, the length of the codon must be determined and the codon sequences must be set. If a codon length of two is used, then using the four naturally occurring bases only 16 possible combinations are available to be used in encoding the library. If the length of the codon is increased to three (the number Nature uses in encoding proteins), the number of possible combinations increases to 64. If the length of the codon is increased to four, the number of possible combinations increases to 256. Other factors to be considered in determining the length of the codon are mismatching, frame-shifting, complexity of library, etc. As the length of the codon is increased up to a certain point the number of mismatches is decreased; however, excessively long codons likely will hybridize despite mismatched base pairs.

Although the length of the codons may vary, the codons may range from 2 to 50 nucleotides, from 2 to 40 nucleotides, from 2 to 30 nucleotides, from 2 to 20 nucleotides, from 2 to 15 nucleotides, from 2 to 10 nucleotides, from 3 to 50 nucleotides, from 3 to 40 nucleotides, from 3 to 30 nucleotides, from 3 to 20 nucleotides, from 3 to 15 nucleotides, from 3 to 10 nucleotides, from 4 to 50 nucleotides, from 4 to 40 nucleotides, from 4 to 30 nucleotides, from 4 to 20 nucleotides, from 4 to 15 nucleotides, from 4 to 10 nucleotides, from 5 to 50 nucleotides, from 5 to 40 nucleotides, from 5 to 30 nucleotides, from 5 to 20 nucleotides, from 5 to 15 nucleotides, from 5 to 10 nucleotides, from 6 to 50 nucleotides, from 6 to 40 nucleotides, from 6 to 30 nucleotides, from 6 to 20 nucleotides, from 6 to 15 nucleotides, from 6 to 10 nucleotides, from 7 to 50 nucleotides, from 7 to 40 nucleotides, from 7 to 30 nucleotides, from 7 to 20 nucleotides, from 7 to 15 nucleotides, from 7 to 10 nucleotides, from 8 to 50 nucleotides, from 8 to 40 nucleotides, from 8 to 30 nucleotides, from 8 to 20 nucleotides, from 8 to 15 nucleotides, from 8 to 10 nucleotides, from 9 to 50 nucleotides, from 9 to 40 nucleotides, from 9 to 30 nucleotides, from 9 to 20 nucleotides, from 9 to 15 nucleotides, from 9 to 10 nucleotides. Codons, however, preferably are 3, 4, 5, 6, 7, 8, 9 or 10 nucleotides in length.

In one embodiment, the set of codons used in the template maximizes the number of mismatches between any two codons within a codon set to ensure that only the proper anti-codons of the transfer units anneal to the codon sites of the template. Furthermore, it is important that the template has mismatches between all the members of one codon set and all the codons of a different codon set to ensure that the anti-codons do not inadvertently bind to the wrong codon set. For example, with regard to the choice of codons n bases in length, each of the codons within a particular codon set should differ with one another by k mismatches, and all of the codons in one codon set should differ by m mismatches with all of the codons in the other codon set. Exemplary values for n, k, and m, for a variety of codon sets suitable for use on a template are published, for example, in Table 1 of U.S. Patent Application Publication No. US-2004/0180412, by Liu et al.

Using an appropriate algorithm, it is possible to generate sets of codons that maximize mismatches between any two codons within the same set, where the codons are n bases long having at least k mismatches between any two codons. Since between any two codons, there must be at least k mismatches, any two subcodons of n−(k−1) bases must have at least one mismatch. This sets an upper limit of 4^{n-k+1} on the size of any (m, k) codon set. Such an algorithm preferably starts with the 4^{n-k+1} possible subcodons of length n−(k−1) and then tests all combinations of adding k−1 bases for those that always maintain k mismatches. All possible (m, k) sets can be generated for n≦6. For n>6, the 4^{n-k+1} upper limits of codons cannot be met and a “full” packing of viable codons is mathematically impossible. In addition to there being at least one mismatch k between codons within the same codon set, there should also be at least one mismatch m between all the codons of one codon set and all the codons of another codon set. Using this approach, different sets of codons can be generated so that no codons are repeated. By way of example, four (n=5, k=3, m=1) sets, each with 64 codons, can be chosen that always have at least one mismatch between any two codons in different sets and at least three mismatches between codons in the same set, as described, for example, in Tables 2-5 of U.S. Patent Application Publication No. US-2004/0180412, by Liu et al. Similarly, four (n=6, k=4, m=2) sets, each with 64 codons, can be chosen that always have at least two mismatches between any two codons in different codon sets and at least four mismatches between codons in the same codon set as described, for example, in Tables 6-9 of U.S. Patent Application Publication No. US-2004/0180412, by Liu et al.

Codons can also be chosen to increase control over the GC content and, therefore, the melting temperature of the codon and anti-codon. Codons sets with a wide range in GC content versus AT content may result in reagents that anneal with different efficiencies due to different melting temperatures. By screening for GC content among different (n, k) sets, the GC content for the codon sets can be optimized. For example, the four (6, 4, 2) codon sets set forth in Tables 6-9 each contain 40 codons with identical GC content (i.e., 50% GC content). By using only these 40 codons at each position, all the reagents in theory will have comparable melting temperatures, removing potential biases in annealing that might otherwise affect library synthesis. Longer codons that maintain a large number of mismatches such as those appropriate for certain applications such as the reaction discovery system can also be chosen using this approach. For example, by combining two (6, 4) sets together while matching low GC to high GC codons, (12, 8) sets with 64 codons all with 50% GC content can be generated for use in reaction discovery selections as well as other application where multiple mismatches might be advantageous. These codons satisfy the requirements for encoding a 30×30 matrix of functional group combinations for reaction discovery.

Although an anti-codon is intended to bind only to a codon, an anti-codon may also bind to an unintended sequence on a template if complementary sequence is present. Thus, an anti-codon may inadvertently bind to a non-codon sequence. Alternatively, an anti-codon might inadvertently bind out-of-frame by annealing in part to one codon and in part to another codon or to a non-codon sequence. Finally, an anti-codon might bind in-frame to an incorrect codon, an issue addressed by the codon sets described above by requiring at least one base difference distinguishing each codon. In Nature, the problems of noncoding sequences and out-of-frame binding are avoided by the ribosome. The nucleic acid-templated methods described herein, however, do not take advantage of the ribosome's fidelity. Therefore, in order to avoid erroneous annealing, the templates can be designed such that sequences complementary to anti-codons are found exclusively at in-frame codon positions. For example, codons can be designed to begin, or end, with a particular base (e.g., “G”). If that base is omitted from all other positions in the template (i.e., all other positions are restricted to T, C, and A), only perfect codon sequences in the template will be at the in-frame codon sequences. Similarly, the codon may be designed to be sufficiently long such that its sequence is unique and does not appear elsewhere in a template.

When the nucleic acid-templated synthesis is used to produce a polymer or a small molecule, spacer sequences may also be placed between the codons to prevent frame shifting. For example, the bases of the template that encode a polymer subunit (the “genetic code” for the polymer) may be chosen from Table A to preclude or minimize the possibility of out-of-frame annealing. These genetic codes reduce undesired frameshifted nucleic acid-templated polymer translation and differ in the range of expected melting temperatures and in the minimum number of mismatches that result during out-of-frame annealing.

TABLE A
Representative Genetic Codes for Nucleic Acid-templated
Polymers That Preclude Out-Of-Frame Annealing

| Sequence | Number of Possible Codons | |
|---|---|---|
| VVNT | 36 | possible codons |
| NVVT | 36 | possible codons |
| SSWT | 8 | possible codons |
| SSST | 8 | possible codons |
| SSNT | 16 | possible codons |
| VNVNT or NVNVT | 144 | possible codons |
| SSSWT or SSWST | 16 | possible codons |
| SNSNT or NSNST | 64 | possible codons |
| SSNWT or SWNST | 32 | possible codons |
| WSNST or NSWST | 32 | possible codons |

where, V = A, C, or G, S = C or G, W = A or T, and N = A, C, G, or T

As in Nature, start and stop codons are useful, particularly in the context of polymer synthesis, to restrict erroneous anti-codon annealing to non-codons and to prevent excessive extension of a growing polymer. For example, a start codon can anneal to a transfer unit bearing a small molecule scaffold or a start monomer unit for use in polymer synthesis; the start monomer unit can be masked by a photolabile protecting group. A stop codon, if used to terminate polymer synthesis, should not conflict with any other codons used in the synthesis and should be of the same general format as the other codons. Generally, a stop codon can encode a monomer unit that terminates polymerization by not providing a reactive group for further attachment. For example, a stop monomer unit may contain a blocked reactive group such as an acetamide rather than a primary amine. In other embodiments, the stop monomer unit can include a biotinylated terminus that terminates the polymerization and facilitates purification of the resulting polymer.

(iii) Template Synthesis

The templates may be synthesized using methodologies well known in the art. For example, the nucleic acid sequence may be prepared using any method known in the art to prepare nucleic acid sequences. These methods include both in vivo and in vitro methods including PCR, plasmid preparation, endonuclease digestion, solid phase synthesis (for example, using an automated synthesizer), in vitro transcription, strand separation, etc. Following synthesis, the template, when desired may be associated (for example, covalently or non covalently coupled) with a reactive unit of interest using standard coupling chemistries known in the art.

An efficient method to synthesize a large variety of templates is to use a “split-pool” technique. The oligonucleotides are synthesized using standard 3′ to 5′ chemistries. First, the constant 3′ end is synthesized. This is then split into n different vessels, where n is the number of different codons to appear at that position in the template. For each vessel, one of the n different codons is synthesized on the (growing) 5′ end of the constant 3′ end. Thus, each vessel contains, from 5′ to 3′, a different codon attached to a constant 3′ end. The n vessels are then pooled, so that a single vessel contains n different codons attached to the constant 3′ end. Any constant bases adjacent the 5′ end of the codon are now synthesized. The pool then is split into m different vessels, where m is the number of different codons to appear at the next (more 5′) position of the template. A different codon is synthesized (at the 5′ end of the growing oligonucleotide) in each of the m vessels. The resulting oligonucleotides are pooled in a single vessel. Splitting, synthesizing, and pooling are repeated as required to synthesize all codons and constant regions in the oligonucleotides.

### II. Transfer Units

A transfer unit comprises an oligonucleotide containing an anti-codon sequence and a reactive unit. The anti-codons are designed to be complementary to the codons present in the template. Accordingly, the sequences used in the template and the codon lengths should be considered when designing the anti-codons. Any molecule complementary to a codon used in the template may be used, including natural or non-natural nucleotides. In certain embodiments, the codons include one or more bases found in nature (i.e., thymidine, uracil, guanidine, cytosine, and adenine). Thus, the anti-codon can include one or more nucleotides normally found in Nature with a base, a sugar, and an optional phosphate group. Alternatively, the bases may be connected via a backbone other than the sugar-phosphate backbone normally found in Nature (e.g., non-natural nucleotides).

As discussed above, the anti-codon is associated with a particular type of reactive unit to form a transfer unit. The reactive unit may represent a distinct entity or may be part of the functionality of the anti-codon unit. In certain embodiments, each anti-codon sequence is associated with one monomer type. For example, the anti-codon sequence ATTAG may be associated with a carbamate residue with an isobutyl side chain, and the anti-codon sequence CATAG may be associated with a carbamate residue with a phenyl side chain. This one-for-one mapping of anti-codon to monomer units allows the decoding of any polymer of the library by sequencing the nucleic acid template used in the synthesis and allows synthesis of the same polymer or a related polymer by knowing the sequence of the original polymer. By changing (e.g., mutating) the sequence of the template, different monomer units may be introduced, thereby allowing the synthesis of related polymers, which can subsequently be selected and evolved. In certain preferred embodiments, several anti-codons may code for one monomer unit as is the case in Nature.

In certain other embodiments, where a small molecule library is to be created rather than a polymer library, the anti-codon generally is associated with a reactive unit or reactant used to modify a small molecule scaffold. In certain embodiments, the reactant is linked to the anti-codon via a linker long enough to allow the reactant to come into reactive proximity with the small molecule scaffold. The linker preferably has a length and composition to permit intramolecular reactions but yet minimize intermolecular reactions. The reactants include a variety of reagents as demonstrated by the wide range of reactions that can be utilized in nucleic acid-templated synthesis and can be any chemical group, catalyst (e.g., organometallic compounds), or reactive moiety (e.g., electrophiles, nucleophiles) known in the chemical arts.

Additionally, the association between the anti-codon and the reactive unit, for example, a monomer unit or reactant, in the transfer unit may be covalent or non-covalent. The association maybe through a covalent bond and, in certain embodiments, the covalent bond may be severable.

Thus, the anti-codon can be associated with the reactant through a linker moiety. The linkage can be cleavable by light, oxidation, hydrolysis, exposure to acid, exposure to base, reduction, etc. Fruchtel et al. (1996) ANGEW. CHEM. INT. ED. ENGL. 35: 17 describes a variety of linkages useful in the practice of the invention. The linker facilitates contact of the reactant with the small molecule scaffold and in certain embodiments, depending on the desired reaction, positions DNA as a leaving group (“autocleavable” strategy), or may link reactive groups to the template via the “scarless” linker strategy (which yields product without leaving behind an additional atom or atoms having chemical functionality), or a “useful scar” strategy (in which a portion of the linker is left behind to be functionalized in subsequent steps following linker cleavage).

With the “autocleavable” linker strategy, the DNA-reactive group bond is cleaved as a natural consequence of the reaction. In the “scarless” linker strategy, DNA-templated reaction of one reactive group is followed by cleavage of the linker attached through a second reactive group to yield products without leaving behind additional atoms capable of providing chemical functionality. Alternatively, a “useful scar” may be utilized on the theory that it may be advantageous to introduce useful atoms and/or chemical groups as a consequence of linker cleavage. In particular, a “useful scar” is left behind following linker cleavage and can be functionalized in subsequent steps.

The anti-codon and the reactive unit (monomer unit) may also be associated through non-covalent interactions such as ionic, electrostatic, hydrogen bonding, van der Waals interactions, hydrophobic interactions, pi-stacking, etc. and combinations thereof. To give but one example, an anti-codon may be linked to biotin, and a monomer unit linked to streptavidin. The propensity of streptavidin to bind biotin leads to the non-covalent association between the anti-codon and the monomer unit to form the transfer unit.

The specific annealing of transfer units to templates permits the use of transfer units at concentrations lower than concentrations used in many traditional organic syntheses. Thus, transfer units can be used at submillimolar concentrations (e.g. less than 100 μM, less than 10 μM, less than 1 μM, less than 100 nM, or less than 10 nM).

### III. Chemical Reactions

A variety of compounds and/or libraries can be prepared using the methods described herein. In certain embodiments, compounds that are not, or do not resemble, nucleic acids or analogs thereof, are synthesized according to the method of the invention. In certain other embodiments, compounds that are not, or do not resemble, proteins, peptides, or analogs thereof, are synthesized according to the method of the invention.

### (i) Coupling Reactions for Small Molecule Synthesis

In some embodiments, it is possible to create compounds such as small molecules using the methods described herein. These small molecules may be like natural products, non-polymeric, and/or non-oligomeric. The substantial interest in small molecules is due in part to their use as the active ingredient in many pharmaceutical preparations although they may also be used, for example, as catalysts, materials, or additives.

In synthesizing small molecules using the method of the present invention, an evolvable template also is provided. The template can include a small molecule scaffold upon which the small molecule is to be built, or a small molecule scaffold may be added to the template. The small molecule scaffold can be any chemical compound with two or more sites for functionalization. For example, the small molecule scaffold can include a ring system (e.g., the ABCD steroid ring system found in cholesterol) with functionalizable groups coupled to the atoms making up the rings. In another example, the small molecule may be the underlying structure of a pharmaceutical agent such as morphine, epothilone or a cephalosporin antibiotic. The sites or groups to be functionalized on the small molecule scaffold may be protected using methods and protecting groups known in the art. The protecting groups used in a small molecule scaffold may be orthogonal to one another so that protecting groups can be removed one at a time.

In this embodiment, the transfer units comprise an anti-codon associated with a reactant or a building block for use in modifying, adding to, or taking away from the small molecule scaffold. The reactants or building blocks may be, for example, electrophiles (e.g., acetyl, amides, acid chlorides, esters, nitrites, imines), nucleophiles (e.g., amines, hydroxyl groups, thiols), catalysts (e.g., organometallic catalysts), or side chains. The transfer units are allowed to contact the template under hydridizing conditions. As a result of oligonucleotide annealing, the attached reactant or building block is allowed to react with a site on the small molecule scaffold. In certain embodiments, protecting groups on the small molecule template are removed one at a time from the sites to be functionalized so that the reactant of the transfer unit will react at only the desired position on the scaffold.

The reaction conditions, linker, reactant, and site to be functionalized are chosen to avoid intermolecular reactions and accelerate intramolecular reactions. Sequential or simultaneous contacting of the template with transfer units can be employed depending on the particular compound to be synthesized. In certain embodiments of special interest, the multi-step synthesis of chemical compounds is provided in which the template is contacted sequentially with two or more transfer units to facilitate multi-step synthesis of complex chemical compounds.

After the sites on the scaffold have been modified, the newly synthesized small molecule remains associated with the template that encoded its synthesis. Decoding the sequence of the template permits the deconvolution of the synthetic history and thereby the structure of the small molecule. The template can also be amplified in order to create more of the desired small molecule and/or the template can be evolved (mutagenized) to create related small molecules. The small molecule can also be cleaved from the template for purification or screening.

### (ii) Coupling Reactions for Polymer Synthesis

In certain embodiments, polymers, specifically unnatural polymers, are prepared according to the method of the present invention. The unnatural polymers that can be created using the inventive method and system include any unnatural polymers. Exemplary unnatural polymers include, but are not limited to, peptide nucleic acid (PNA) polymers, polycarbamates, polyureas, polyesters, polyacrylate, polyalkylene (e.g., polyethylene, polypropylene), polycarbonates, polypeptides with unnatural stereochemistry, polypeptides with unnatural amino acids, and combination thereof. In certain embodiments, the polymers comprise at least 10, 25, 75, 100, 125, 150 monomer units or more. The polymers synthesized using the inventive system may be used, for example, as catalysts, pharmaceuticals, metal chelators, or catalysts.

In preparing certain unnatural polymers, the monomer units attached to the anti-codons may be any monomers or oligomers capable of being joined together to form a polymer. The monomer units may be, for example, carbamates, D-amino acids, unnatural amino acids, PNAs, ureas, hydroxy acids, esters, carbonates, acrylates, or ethers. In certain embodiments, the monomer units have two reactive groups used to link the monomer unit into the growing polymer chain. Preferably, the two reactive groups are not the same so that the monomer unit may be incorporated into the polymer in a directional sense, for example, at one end may be an electrophile and at the other end a nucleophile. Reactive groups may include, but are not limited to, esters, amides, carboxylic acids, activated carbonyl groups, acid chlorides, amines, hydroxyl groups, and thiols. In certain embodiments, the reactive groups are masked or protected (Greene et al. (1999) PROTECTIVE GROUPS IN ORGANIC SYNTHESIS 3rd Edition, Wiley) so that polymerization may not take place until a desired time when the reactive groups are deprotected. Once the monomer units are assembled along the nucleic acid template, initiation of the polymerization sequence results in a cascade of polymerization and deprotection steps wherein the polymerization step results in deprotection of a reactive group to be used in the subsequent polymerization step.

The monomer units to be polymerized can include two or more monomers depending on the geometry along the nucleic acid template. The monomer units to be polymerized must be able to stretch along the nucleic acid template and particularly across the distance spanned by its encoding anti-codon and optional spacer sequence. In certain embodiments, the monomer unit actually comprises two monomers, for example, a dicarbamate, a diurea, or a dipeptide. In yet other embodiments, the monomer unit comprises three or more monomers.

The monomer units may contain any chemical groups known in the art. Reactive chemical groups especially those that would interfere with polymerization, hybridization, etc., are preferably masked using known protecting groups (Greene et al. (1999) supra). In general, the protecting groups used to mask these reactive groups are orthogonal to those used in protecting the groups used in the polymerization steps.

It has been discovered that, under certain circumstances, the type of chemical reaction may affect the fidelity of the polymerization process. For example, distance independent chemical reactions (for example, reactions that occur efficiently when the reactive units are spaced apart by intervening bases, for example, amine acylation reactions) may result in the spurious incorporation of the wrong monomers at a particular position of a polymer chain. In contrast, by choosing chemical reactions for template mediated syntheses that are distance dependent (for example, reactions that become inefficient the further the reactive units are spaced part via intervening bases, for example, reductive amination reactions), it is possible control the fidelity of the polymerization process.

(iii) Functional Group Transformations

Nucleic acid-templated synthesis can be used to effect functional group transformations that either (i) unmask or (ii) interconvert functionality used in coupling reactions. By exposing or creating a reactive group within a sequence-programmed subset of a library, nucleic acid-templated functional group interconversions permit the generation of library diversity by sequential unmasking. The sequential unmasking approach offers the major advantage of enabling reactants that would normally lack the ability to be linked to a nucleic acid (for example, simple alkyl halides) to contribute to library diversity by reacting with a sequence-specified subset of templates in an intermolecular, non-templated reaction mode. This advantage significantly increases the types of structures that can be generated.

One embodiment of the invention involves deprotection or unmasking of functional groups present in a reactive unit. According to this embodiment, a nucleic acid-template is associated with a reactive unit that contains a protected functional group. A transfer unit, comprising an oligonucleotide complimentary to the template codon region and a reagent capable of removing the protecting group, is annealed to the template, and the reagent reacts with the protecting group, removing it from the reactive unit. To further functionalize the reactive unit, the exposed functional group then is subjected to a reagent not linked to a nucleic acid. In some embodiments, the reactive unit contains two or more protected functional groups. In still other embodiments, the protecting groups are orthogonal protecting groups that are sequentially removed by iterated annealing with reagents linked to transfer units.

Another embodiment of the invention involves interconversions of functional groups present on a reactive unit. According to this embodiment, a transfer unit associated with a reagent that can catalyze a reaction is annealed to a template bearing the reactive unit. A reagent not linked to a nucleic acid is added to the reaction, and the transfer unit reagent catalyzes the reaction between the unlinked reagent and the reactive unit, yielding a newly functionalized reactive unit. In some embodiments, the reactive unit contains two or more functional groups which are sequentially interconverted by iterative exposure to different transfer unit-bound reagents.

### (iv) Reaction Conditions

Nucleic acid-templated reactions can occur in aqueous or non-aqueous (i.e., organic) solutions, or a mixture of one or more aqueous and non-aqueous solutions. In aqueous solutions, reactions can be performed at pH ranges from about 2 to about 12, or preferably from about 2 to about 10, or more preferably from about 4 to about 10. The reactions used in DNA-templated chemistry preferably should not require very basic conditions (e.g., pH>12, pH>10) or very acidic conditions (e.g., pH<1, pH<2, pH<4), because extreme conditions may lead to degradation or modification of the nucleic acid template and/or molecule (for example, the polymer, or small molecule) being synthesized. The aqueous solution can contain one or more inorganic salts, including, but not limited to, NaCl, Na₂SO₄, KCl, Mg⁺², Mn⁺², etc., at various concentrations.

Any organic solvent that facilitates nucleic acid-templated chemistry may be used in the present invention. Exemplary solvents include CH₃CN, DMF, THF, organic alcohols (e.g., CH₃OH, C₂H₅OH)CH₂Cl₂, CCl₄, CHCl₃, toluene, benzene, diethyl ether, glyme, hexanes, and DMSO. The weight percentage of organic solvent in a solution may be 100% or the weight percentage may be 99%, 98%, 97%, 95%, 90%, 80%, 70%, 60%, 50% organic solvent or more or less than any of the foregoing percentages. The weight percentage of water in a solution may be 0% or may be 1%, 2%, 3%, 5%, 10%, 20%, 30%, 40% water, or more or less than any of the foregoing percentages.

To facilitate a nucleic acid-templated reaction, the template and/or the transfer units may be solublized by one or more quaternary ammonium ions. Exemplary ions include ions of the formula ⁺NR₁R₂R₃R₄. Each of the R's may be the same or different unsubstituted or substituted alkyl groups, e.g., alkyl groups with 1, 2, 3, 4, 5, 6, 7, 8, 9, 10, 11, 12, 13, 14, 15, 16, 17, 18, 19, or more carbon atoms. The alkyl groups may be branched with one, two three or more substitutions. To permit quantitative dissolution of reaction components in organic solvents, quaternized ammonium salts, such as, for example, long chain tetraalkylammonium salts, can be added (Jost et al. (1989) NUCLEIC ACIDS RES. 17: 2143; Mel'nikov et al. (1999) LANGMUIR 15: 1923-1928).

Nucleic acid-templated reactions may require a catalyst, such as, for example, homogeneous, heterogeneous, phase transfer, and asymmetric catalysis. In other embodiments, a catalyst is not required. The presence of additional, accessory reagents not linked to a nucleic acid are preferred in some embodiments. Useful accessory reagents can include, for example, oxidizing agents (e.g., NalO₄); reducing agents (e.g., NaCNBH₃); activating reagents (e.g., EDC, NHS, and sulfo-NHS); transition metals such as nickel (e.g., Ni(NO₃)₂), rhodium (e.g. RhCl₃), ruthenium (e.g. RuCl₃), copper (e.g. Cu(NO₃)₂), cobalt (e.g. COCl₂), iron (e.g. Fe(NO₃)₃), osmium (e.g. OSO₄), titanium (e.g. TiCl₄ or titanium tetraisopropoxide), palladium (e.g. NaPdCl₄), or Ln; transition metal ligands (e.g., phosphines, amines, and halides); Lewis acids; and Lewis bases.

Reaction conditions preferably are optimized to suit the nature of the reactive units and oligonucleotides used.

### (v) Classes of Chemical Reactions

Known chemical reactions for synthesizing polymers, small molecules, or other molecules can be used in nucleic acid-templated reactions. Thus, reactions such as those listed in March's Advanced Organic Chemistry, Organic Reactions, Organic Syntheses, organic text books, journals such as Journal of the American Chemical Society, Journal of Organic Chemistry, Tetrahedron, etc., and Carruther's Some Modern Methods of Organic Chemistry can be used. The chosen reactions preferably are compatible with nucleic acids such as DNA or RNA or are compatible with the modified nucleic acids used as the template.

Reactions useful in nucleic-acid templated chemistry include, for example, substitution reactions, carbon-carbon bond forming reactions, elimination reactions, acylation reactions, and addition reactions. An illustrative but not exhaustive list of aliphatic nucleophilic substitution reactions useful in the present invention includes, for example, S_{N}2 reactions, S_{N}1 reactions, S_{N}i reactions, allylic rearrangements, nucleophilic substitution at an aliphatic trigonal carbon, and nucleophilic substation at a vinylic carbon.

Specific aliphatic nucleophilic substitution reactions with oxygen nucleophiles include, for example, hydrolysis of alkyl halides, hydrolysis of gen-dihalides, hydrolysis of 1,1,1-trihalides, hydrolysis of alkyl esters or inorganic acids, hydrolysis of diazo ketones, hydrolysis of acetal and enol ethers, hydrolysis of epoxides, hydrolysis of acyl halides, hydrolysis of anhydrides, hydrolysis of carboxylic esters, hydrolysis of amides, alkylation with alkyl halides (Williamson Reaction), epoxide formation, alkylation with inorganic esters, alkylation with diazo compounds, dehydration of alcohols, transetherification, alcoholysis of epoxides, alkylation with onium salts, hydroxylation of silanes, alcoholysis of acyl halides, alcoholysis of anhydrides, esterfication of carboxylic acids, alcoholysis of carboxylic esters (transesterfication), alcoholysis of amides, alkylation of carboxylic acid salts, cleavage of ether with acetic anhydride, alkylation of carboxylic acids with diazo compounds, acylation of carboxylic acids with acyl halides, acylation of carboxylic acids with carboxylic acids, formation of oxonium salts, preparation of peroxides and hydroperoxides, preparation of inorganic esters (e.g., nitrites, nitrates, sulfonates), preparation of alcohols from amines, and preparation of mixed organic-inorganic anhydrides.

Specific aliphatic nucleophilic substitution reactions with sulfur nucleophiles, which tend to be better nucleophiles than their oxygen analogs, include, for example, attack by SH at an alkyl carbon to form thiols, attack by S at an alkyl carbon to form thioethers, attack by SH or SR at an acyl carbon, formation of disulfides, formation of Bunte salts, alkylation of sulfinic acid salts, and formation of alkyl thiocyanates.

Aliphatic nucleophilic substitution reactions with nitrogen nucleophiles include, for example, alkylation of amines, N-arylation of amines, replacement of a hydroxy by an amino group, transamination, transamidation, alkylation of amines with diazo compounds, amination of epoxides, amination of oxetanes, amination of aziridines, amination of alkanes, formation of isocyanides, acylation of amines by acyl halides, acylation of amines by anhydrides, acylation of amines by carboxylic acids, acylation of amines by carboxylic esters, acylation of amines by amides, acylation of amines by other acid derivatives, N-alkylation or N-arylation of amides and imides, N-acylation of amides and imides, formation of aziridines from epoxides, formation of nitro compounds, formation of azides, formation of isocyanates and isothiocyanates, and formation of azoxy compounds.

Aliphatic nucleophilic substitution reactions with halogen nucleophiles include, for example, attack at an alkyl carbon, halide exchange, formation of alkyl halides from esters of sulfuric and sulfonic acids, formation of alkyl halides from alcohols, formation of alkyl halides from ethers, formation of halohydrins from epoxides, cleavage of carboxylic esters with lithium iodide, conversion of diazo ketones to α-halo ketones, conversion of amines to halides, conversion of tertiary amines to cyanamides (the von Braun reaction), formation of acyl halides from carboxylic acids, and formation of acyl halides from acid derivatives.

Aliphatic nucleophilic substitution reactions using hydrogen as a nucleophile include, for example, reduction of alkyl halides, reduction of tosylates, other sulfonates, and similar compounds, hydrogenolysis of alcohols, hydrogenolysis of esters (Barton-McCombie reaction), hydrogenolysis of nitriles, replacement of alkoxyl by hydrogen, reduction of epoxides, reductive cleavage of carboxylic esters, reduction of a C—N bond, desulfurization, reduction of acyl halides, reduction of carboxylic acids, esters, and anhydrides to aldehydes, and reduction of amides to aldehydes.

Although certain carbon nucleophiles may be too nucleophilic and/or basic to be used in certain embodiments of the invention, aliphatic nucleophilic substitution reactions using carbon nucleophiles include, for example, coupling with silanes, coupling of alkyl halides (the Wurtz reaction), the reaction of alkyl halides and sulfonate esters with Group I (I A) and II (II A) organometallic reagents, reaction of alkyl halides and sulfonate esters with organocuprates, reaction of alkyl halides and sulfonate esters with other organometallic reagents, allylic and propargylic coupling with a halide substrate, coupling of organometallic reagents with esters of sulfuric and sulfonic acids, sulfoxides, and sulfones, coupling involving alcohols, coupling of organometallic reagents with carboxylic esters, coupling of organometallic reagents with compounds containing an ester linkage, reaction of organometallic reagents with epoxides, reaction of organometallics with aziridine, alkylation at a carbon bearing an active hydrogen, alkylation of ketones, nitriles, and carboxylic esters, alkylation of carboxylic acid salts, alkylation at a position a to a heteroatom (alkylation of 1,3-dithianes), alkylation of dihydro-1,3-oxazine (the Meyers synthesis of aldehydes, ketones, and carboxylic acids), alkylation with trialkylboranes, alkylation at an alkynyl carbon, preparation of nitriles, direct conversion of alkyl halides to aldehydes and ketones, conversion of alkyl halides, alcohols, or alkanes to carboxylic acids and their derivatives, the conversion of acyl halides to ketones with organometallic compounds, the conversion of anhydrides, carboxylic esters, or amides to ketones with organometallic compounds, the coupling of acyl halides, acylation at a carbon bearing an active hydrogen, acylation of carboxylic esters by carboxylic esters (the Claisen and Dieckmann condensation), acylation of ketones and nitriles with carboxylic esters, acylation of carboxylic acid salts, preparation of acyl cyanides, and preparation of diazo ketones, ketonic decarboxylation.

Reactions which involve nucleophilic attack at a sulfonyl sulfur atom may also be used in the present invention and include, for example, hydrolysis of sulfonic acid derivatives (attack by OH), formation of sulfonic esters (attack by OR), formation of sulfonamides (attack by nitrogen), formation of sulfonyl halides (attack by halides), reduction of sulfonyl chlorides (attack by hydrogen), and preparation of sulfones (attack by carbon).

Aromatic electrophilic substitution reactions may also be used in nucleotide-templated chemistry. Hydrogen exchange reactions are examples of aromatic electrophilic substitution reactions that use hydrogen as the electrophile. Aromatic electrophilic substitution reactions which use nitrogen electrophiles include, for example, nitration and nitro-de-hydrogenation, nitrosation of nitroso-de-hydrogenation, diazonium coupling, direct introduction of the diazonium group, and amination or amino-de-hydrogenation. Reactions of this type with sulfur electrophiles include, for example, sulfonation, sulfo-de-hydrogenation, halosulfonation, halosulfo-de-hydrogenation, sulfurization, and sulfonylation. Reactions using halogen electrophiles include, for example, halogenation, and halo-de-hydrogenation. Aromatic electrophilic substitution reactions with carbon electrophiles include, for example, Friedel-Crafts alkylation, alkylation, alkyl-de-hydrogenation, Friedel-Crafts arylation (the Scholl reaction), Friedel-Crafts acylation, formylation with disubstituted formamides, formylation with zinc cyanide and HCl (the Gatterman reaction), formylation with chloroform (the Reimer-Tiemann reaction), other formylations, formyl-de-hydrogenation, carboxylation with carbonyl halides, carboxylation with carbon dioxide (the Kolbe-Schmitt reaction), amidation with isocyanates, N-alkylcarbamoyl-de-hydrogenation, hydroxyalkylation, hydroxyalkyl-de-hydrogenation, cyclodehydration of aldehydes and ketones, haloalkylation, halo-de-hydrogenation, aminoalkylation, amidoalkylation, dialkylaminoalkylation, dialkylamino-de-hydrogenation, thioalkylation, acylation with nitriles (the Hoesch reaction), cyanation, and cyano-de-hydrogenation. Reactions using oxygen electrophiles include, for example, hydroxylation and hydroxy-de-hydrogenation.

Rearrangement reactions include, for example, the Fries rearrangement, migration of a nitro group, migration of a nitroso group (the Fischer-Hepp Rearrangement), migration of an arylazo group, migration of a halogen (the Orton rearrangement), migration of an alkyl group, etc. Other reaction on an aromatic ring include the reversal of a Friedel-Crafts alkylation, decarboxylation of aromatic aldehydes, decarboxylation of aromatic acids, the Jacobsen reaction, deoxygenation, desulfonation, hydro-de-sulfonation, dehalogenation, hydro-de-halogenation, and hydrolysis of organometallic compounds.

Aliphatic electrophilic substitution reactions are also useful. Reactions using the S_{E}1, S_{E}2 (front), S_{E}2 (back), S_{E}i, addition-elimination, and cyclic mechanisms can be used in the present invention. Reactions of this type with hydrogen as the leaving group include, for example, hydrogen exchange (deuterio-de-hydrogenation, deuteriation), migration of a double bond, and keto-enol tautomerization. Reactions with halogen electrophiles include, for example, halogenation of aldehydes and ketones, halogenation of carboxylic acids and acyl halides, and halogenation of sulfoxides and sulfones. Reactions with nitrogen electrophiles include, for example, aliphatic diazonium coupling, nitrosation at a carbon bearing an active hydrogen, direct formation of diazo compounds, conversion of amides to α-azido amides, direct amination at an activated position, and insertion by nitrenes. Reactions with sulfur or selenium electrophiles include, for example, sulfenylation, sulfonation, and selenylation of ketones and carboxylic esters. Reactions with carbon electrophiles include, for example, acylation at an aliphatic carbon, conversion of aldehydes to β-keto esters or ketones, cyanation, cyano-de-hydrogenation, alkylation of alkanes, the Stork enamine reaction, and insertion by carbenes. Reactions with metal electrophiles include, for example, metalation with organometallic compounds, metalation with metals and strong bases, and conversion of enolates to silyl enol ethers. Aliphatic electrophilic substitution reactions with metals as leaving groups include, for example, replacement of metals by hydrogen, reactions between organometallic reagents and oxygen, reactions between organometallic reagents and peroxides, oxidation of trialkylboranes to borates, conversion of Grignard reagents to sulfur compounds, halo-de-metalation, the conversion of organometallic compounds to amines, the conversion of organometallic compounds to ketones, aldehydes, carboxylic esters and amides, cyano-de-metalation, transmetalation with a metal, transmetalation with a metal halide, transmetalation with an organometallic compound, reduction of alkyl halides, metallo-de-halogenation, replacement of a halogen by a metal from an organometallic compound, decarboxylation of aliphatic acids, cleavage of alkoxides, replacement of a carboxyl group by an acyl group, basic cleavage of β-keto esters and β-diketones, haloform reaction, cleavage of non-enolizable ketones, the Haller-Bauer reaction, cleavage of alkanes, decyanation, and hydro-de-cyanation. Electrophilic substitution reactions at nitrogen include, for example, diazotization, conversion of hydrazines to azides, N-nitrosation, N-nitroso-de-hydrogenation, conversion of amines to azo compounds, N-halogenation, N-halo-de-hydrogenation, reactions of amines with carbon monoxide, and reactions of amines with carbon dioxide.

Aromatic nucleophilic substitution reactions may also be used in the present invention. Reactions proceeding via the S_{N}Ar mechanism, the S_{N}1 mechanism, the benzyne mechanism, the S_{RN}1 mechanism, or other mechanism, for example, can be used. Aromatic nucleophilic substitution reactions with oxygen nucleophiles include, for example, hydroxy-de-halogenation, alkali fusion of sulfonate salts, and replacement of OR or OAr. Reactions with sulfur nucleophiles include, for example, replacement by SH or SR. Reactions using nitrogen nucleophiles include, for example, replacement by NH₂, NHR, or NR₂, and replacement of a hydroxy group by an amino group. Reactions with halogen nucleophiles include, for example, the introduction halogens. Aromatic nucleophilic substitution reactions with hydrogen as the nucleophile include, for example, reduction of phenols and phenolic esters and ethers, and reduction of halides and nitro compounds. Reactions with carbon nucleophiles include, for example, the Rosenmund-von Braun reaction, coupling of organometallic compounds with aryl halides, ethers, and carboxylic esters, arylation at a carbon containing an active hydrogen, conversions of aryl substrates to carboxylic acids, their derivatives, aldehydes, and ketones, and the Ullmann reaction. Reactions with hydrogen as the leaving group include, for example, alkylation, arylation, and amination of nitrogen heterocycles. Reactions with N₂⁺ as the leaving group include, for example, hydroxy-de-diazoniation, replacement by sulfur-containing groups, iodo-de-diazoniation, and the Schiemann reaction. Rearrangement reactions include, for example, the von Richter rearrangement, the Sommelet-Hauser rearrangement, rearrangement of aryl hydroxylamines, and the Smiles rearrangement.

Reactions involving free radicals can also be used, although the free radical reactions used in nucleotide-templated chemistry should be carefully chosen to avoid modification or cleavage of the nucleotide template. With that limitation, free radical substitution reactions can be used in the present invention. Particular free radical substitution reactions include, for example, substitution by halogen, halogenation at an alkyl carbon, allylic halogenation, benzylic halogenation, halogenation of aldehydes, hydroxylation at an aliphatic carbon, hydroxylation at an aromatic carbon, oxidation of aldehydes to carboxylic acids, formation of cyclic ethers, formation of hydroperoxides, formation of peroxides, acyloxylation, acyloxy-de-hydrogenation, chlorosulfonation, nitration of alkanes, direct conversion of aldehydes to amides, amidation and amination at an alkyl carbon, simple coupling at a susceptible position, coupling of alkynes, arylation of aromatic compounds by diazonium salts, arylation of activated alkenes by diazonium salts (the Meerwein arylation), arylation and alkylation of alkenes by organopalladium compounds (the Heck reaction), arylation and alkylation of alkenes by vinyltin compounds (the Stille reaction), alkylation and arylation of aromatic compounds by peroxides, photochemical arylation of aromatic compounds, alkylation, acylation, and carbalkoxylation of nitrogen heterocycles Particular reactions in which N₂⁺ is the leaving group include, for example, replacement of the diazonium group by hydrogen, replacement of the diazonium group by chlorine or bromine, nitro-de-diazoniation, replacement of the diazonium group by sulfur-containing groups, aryl dimerization with diazonium salts, methylation of diazonium salts, vinylation of diazonium salts, arylation of diazonium salts, and conversion of diazonium salts to aldehydes, ketones, or carboxylic acids. Free radical substitution reactions with metals as leaving groups include, for example, coupling of Grignard reagents, coupling of boranes, and coupling of other organometallic reagents. Reaction with halogen as the leaving group are included. Other free radical substitution reactions with various leaving groups include, for example, desulfurization with Raney Nickel, conversion of sulfides to organolithium compounds, decarboxylative dimerization (the Kolbe reaction), the Hunsdiecker reaction, decarboxylative allylation, and decarbonylation of aldehydes and acyl halides.

Reactions involving additions to carbon-carbon multiple bonds are also used in nucleotide-templated chemistry. Any mechanism may be used in the addition reaction including, for example, electrophilic addition, nucleophilic addition, free radical addition, and cyclic mechanisms. Reactions involving additions to conjugated systems can also be used. Addition to cyclopropane rings can also be utilized. Particular reactions include, for example, isomerization, addition of hydrogen halides, hydration of double bonds, hydration of triple bonds, addition of alcohols, addition of carboxylic acids, addition of H₂S and thiols, addition of ammonia and amines, addition of amides, addition of hydrazoic acid, hydrogenation of double and triple bonds, other reduction of double and triple bonds, reduction of the double and triple bonds of conjugated systems, hydrogenation of aromatic rings, reductive cleavage of cyclopropanes, hydroboration, other hydrometalations, addition of alkanes, addition of alkenes and/or alkynes to alkenes and/or alkynes (e.g., pi-cation cyclization reactions, hydro-alkenyl-addition), ene reactions, the Michael reaction, addition of organometallics to double and triple bonds not conjugated to carbonyls, the addition of two alkyl groups to an alkyne, 1,4-addition of organometallic compounds to activated double bonds, addition of boranes to activated double bonds, addition of tin and mercury hydrides to activated double bonds, acylation of activated double bonds and of triple bonds, addition of alcohols, amines, carboxylic esters, aldehydes, etc., carbonylation of double and triple bonds, hydrocarboxylation, hydroformylation, addition of aldehydes, addition of HCN, addition of silanes, radical addition, radical cyclization, halogenation of double and triple bonds (addition of halogen, halogen), halolactonization, halolactamization, addition of hypohalous acids and hypohalites (addition of halogen, oxygen), addition of sulfur compounds (addition of halogen, sulfur), addition of halogen and an amino group (addition of halogen, nitrogen), addition of NOX and NO₂X (addition of halogen, nitrogen), addition of XN₃ (addition of halogen, nitrogen), addition of alkyl halides (addition of halogen, carbon), addition of acyl halides (addition of halogen, carbon), hydroxylation (addition of oxygen, oxygen) (e.g., asymmetric dihydroxylation reaction with OSO₄), dihydroxylation of aromatic rings, epoxidation (addition of oxygen, oxygen) (e.g., Sharpless asymmetric epoxidation), photooxidation of dienes (addition of oxygen, oxygen), hydroxysulfenylation (addition of oxygen, sulfur), oxyamination (addition of oxygen, nitrogen), diamination (addition of nitrogen, nitrogen), formation of aziridines (addition of nitrogen), aminosulfenylation (addition of nitrogen, sulfur), acylacyloxylation and acylamidation (addition of oxygen, carbon or nitrogen, carbon), 1,3-dipolar addition (addition of oxygen, nitrogen, carbon), Diels-Alder reaction, heteroatom Diels-Alder reaction, all carbon 3+2 cycloadditions, dimerization of alkenes, the addition of carbenes and carbenoids to double and triple bonds, trimerization and tetramerization of alkynes, and other cycloaddition reactions.

In addition to reactions involving additions to carbon-carbon multiple bonds, addition reactions to carbon-hetero multiple bonds can be used in nucleotide-templated chemistry. Exemplary reactions include, for example, the addition of water to aldehydes and ketones (formation of hydrates), hydrolysis of carbon-nitrogen double bond, hydrolysis of aliphatic nitro compounds, hydrolysis of nitriles, addition of alcohols and thiols to aldehydes and ketones, reductive alkylation of alcohols, addition of alcohols to isocyanates, alcoholysis of nitriles, formation of xanthates, addition of H₂S and thiols to carbonyl compounds, formation of bisulfite addition products, addition of amines to aldehydes and ketones, addition of amides to aldehydes, reductive alkylation of ammonia or amines, the Mannich reaction, the addition of amines to isocyanates, addition of ammonia or amines to nitriles, addition of amines to carbon disulfide and carbon dioxide, addition of hydrazine derivative to carbonyl compounds, formation of oximes, conversion of aldehydes to nitriles, formation of gem-dihalides from aldehydes and ketones, reduction of aldehydes and ketones to alcohols, reduction of the carbon-nitrogen double bond, reduction of nitriles to amines, reduction of nitriles to aldehydes, addition of Grignard reagents and organolithium reagents to aldehydes and ketones, addition of other organometallics to aldehydes and ketones, addition of trialkylallylsilanes to aldehydes and ketones, addition of conjugated alkenes to aldehydes (the Baylis-Hillman reaction), the Reformatsky reaction, the conversion of carboxylic acid salts to ketones with organometallic compounds, the addition of Grignard reagents to acid derivatives, the addition of organometallic compounds to CO₂ and CS₂, addition of organometallic compounds to C═N compounds, addition of carbenes and diazoalkanes to C═N compounds, addition of Grignard reagents to nitriles and isocyanates, the Aldol reaction, Mukaiyama Aldol and related reactions, Aldol-type reactions between carboxylic esters or amides and aldehydes or ketones, the Knoevenagel reaction (e.g., the Nef reaction, the Favorskii reaction), the Peterson alkenylation reaction, the addition of active hydrogen compounds to CO₂ and CS₂, the Perkin reaction, Darzens glycidic ester condensation, the Tollens' reaction, the Wittig reaction, the Tebbe alkenylation, the Petasis alkenylation, alternative alkenylations, the Thorpe reaction, the Thorpe-Ziegler reaction, addition of silanes, formation of cyanohydrins, addition of HCN to C═N and C═N bonds, the Prins reaction, the benzoin condensation, addition of radicals to C═O, C═S, C═N compounds, the Ritter reaction, acylation of aldehydes and ketones, addition of aldehydes to aldehydes, the addition of isocyanates to isocyanates (formation of carbodiimides), the conversion of carboxylic acid salts to nitriles, the formation of epoxides from aldehydes and ketones, the formation of episulfides and episulfones, the formation of β-lactones and oxetanes (e.g., the Patemo-Btichi reaction), the formation of β-lactams, etc. Reactions involving addition to isocyanides include the addition of water to isocyanides, the Passerini reaction, the Ug reaction, and the formation of metalated aldimines.

Elimination reactions, including α, β, and γ eliminations, as well as extrusion reactions, can be performed using nucleotide-templated chemistry, although the strength of the reagents and conditions employed should be considered. Preferred elimination reactions include reactions that go by E1, E2, E1cB, or E2C mechanisms. Exemplary reactions include, for example, reactions in which hydrogen is removed from one side (e.g., dehydration of alcohols, cleavage of ethers to alkenes, the Chugaev reaction, ester decomposition, cleavage of quarternary ammonium hydroxides, cleavage of quaternary ammonium salts with strong bases, cleavage of amine oxides, pyrolysis of keto-ylids, decomposition of toluene-p-sulfonylhydrazones, cleavage of sulfoxides, cleavage of selenoxides, cleavage of sulfornes, dehydrogalogenation of alkyl halides, dehydrohalogenation of acyl halides, dehydrohalogenation of sulfonyl halides, elimination of boranes, conversion of alkenes to alkynes, decarbonylation of acyl halides), reactions in which neither leaving atom is hydrogen (e.g., deoxygenation of vicinal diols, cleavage of cyclic thionocarbonates, conversion of epoxides to episulfides and alkenes, the Ramberg-Backlund reaction, conversion of aziridines to alkenes, dehalogenation of vicinal dihalides, dehalogenation of α-halo acyl halides, and elimination of a halogen and a hetero group), fragmentation reactions (i.e., reactions in which carbon is the positive leaving group or the electrofuge, such as, for example, fragmentation of γ-amino and γ-hydroxy halides, fragmentation of 1,3-diols, decarboxylation of β-hydroxy carboxylic acids, decarboxylation of β-lactones, fragmentation of α,β-epoxy hydrazones, elimination of CO from briged bicyclic compounds, and elimination of CO₂ from bridged bicyclic compounds), reactions in which C—N or C═N bonds are formed (e.g., dehydration of aldoximes or similar compounds, conversion of ketoximes to nitriles, dehydration of unsubstituted amides, and conversion of N-alkylformamides to isocyanides), reactions in which C═O bonds are formed (e.g., pyrolysis of β-hydroxy alkenes), and reactions in which N═N bonds are formed (e.g., eliminations to give diazoalkenes). Extrusion reactions include, for example, extrusion of N₂ from pyrazolines, extrusion of N₂ from pyrazoles, extrusion of N₂ from triazolines, extrusion of CO, extrusion of CO₂, extrusion of SO₂, the Story synthesis, and alkene synthesis by twofold extrusion.

Rearrangements, including, for example, nucleophilic rearrangements, electrophilic rearrangements, prototropic rearrangements, and free-radical rearrangements, can also be performed using nucleotide-templated chemistry. Both 1,2 rearrangements and non-1,2 rearrangements can be performed. Exemplary reactions include, for example, carbon-to-carbon migrations of R, H, and Ar (e.g., Wagner-Meerwein and related reactions, the Pinacol rearrangement, ring expansion reactions, ring contraction reactions, acid-catalyzed rearrangements of aldehydes and ketones, the dienone-phenol rearrangement, the Favorskii rearrangement, the Arndt-Eistert synthesis, homologation of aldehydes, and homologation of ketones), carbon-to-carbon migrations of other groups (e.g., migrations of halogen, hydroxyl, amino, etc.; migration of boron; and the Neber rearrangement), carbon-to-nitrogen migrations of R and Ar (e.g., the Hofmann rearrangement, the Curtius rearrangement, the Lossen rearrangement, the Schmidt reaction, the Beckman rearrangement, the Stieglits rearrangement, and related rearrangements), carbon-to-oxygen migrations of R and Ar (e.g., the Baeyer-Villiger rearrangement and rearrangement of hydroperoxides), nitrogen-to-carbon, oxygen-to-carbon, and sulfur-to-carbon migration (e.g., the Stevens rearrangement, and the Wittig rearrangement), boron-to-carbon migrations (e.g., conversion of boranes to alcohols (primary or otherwise), conversion of boranes to aldehydes, conversion of boranes to carboxylic acids, conversion of vinylic boranes to alkenes, formation of alkynes from boranes and acetylides, formation of alkenes from boranes and acetylides, and formation of ketones from boranes and acetylides), electrocyclic rearrangements (e.g., of cyclobutenes and 1,3-cyclohexadienes, or conversion of stilbenes to phenanthrenes), sigmatropic rearrangements (e.g., (1,j) sigmatropic migrations of hydrogen, (1,j) sigmatropic migrations of carbon, conversion of vinylcyclopropanes to cyclopentenes, the Cope rearrangement, the Claisen rearrangement, the Fischer indole synthesis, (2,3) sigmatropic rearrangements, and the benzidine rearrangement), other cyclic rearrangements (e.g., metathesis of alkenes, the di-π-methane and related rearrangements, and the Hofmann-Löffler and related reactions), and non-cyclic rearrangements (e.g., hydride shifts, the Chapman rearrangement, the Wallach rearrangement, and dyotropic rearrangements).

Oxidative and reductive reactions may also be performed using nucleotide-templated chemistry. Exemplary reactions may involve, for example, direct electron transfer, hydride transfer, hydrogen-atom transfer, formation of ester intermediates, displacement mechanisms, or addition-elimination mechanisms. Exemplary oxidations include, for example, eliminations of hydrogen (e.g., aromatization of six-membered rings, dehydrogenations yielding carbon-carbon double bonds, oxidation or dehydrogenation of alcohols to aldehydes and ketones, oxidation of phenols and aromatic amines to quinones, oxidative cleavage of ketones, oxidative cleavage of aldehydes, oxidative cleavage of alcohols, ozonolysis, oxidative cleavage of double bonds and aromatic rings, oxidation of aromatic side chains, oxidative decarboxylation, and bisdecarboxylation), reactions involving replacement of hydrogen by oxygen (e.g., oxidation of methylene to carbonyl, oxidation of methylene to OH, CO₂R, or OR, oxidation of arylmethanes, oxidation of ethers to carboxylic esters and related reactions, oxidation of aromatic hydrocarbons to quinones, oxidation of amines or nitro compounds to aldehydes, ketones, or dihalides, oxidation of primary alcohols to carboxylic acids or carboxylic esters, oxidation of alkenes to aldehydes or ketones, oxidation of amines to nitroso compounds and hydroxylamines, oxidation of primary amines, oximes, azides, isocyanates, or notroso compounds, to nitro compounds, oxidation of thiols and other sulfur compounds to sulfonic acids), reactions in which oxygen is added to the substrate (e.g., oxidation of alkynes to α-diketones, oxidation of tertiary amines to amine oxides, oxidation of thioesters to sulfoxides and sulfones, and oxidation of carboxylic acids to peroxy acids), and oxidative coupling reactions (e.g., coupling involving carbanoins, dimerization of silyl enol ethers or of lithium enolates, and oxidation of thiols to disulfides).

Exemplary reductive reactions include, for example, reactions involving replacement of oxygen by hydrogen (e.g., reduction of carbonyl to methylene in aldehydes and ketones, reduction of carboxylic acids to alcohols, reduction of amides to amines, reduction of carboxylic esters to ethers, reduction of cyclic anhydrides to lactones and acid derivatives to alcohols, reduction of carboxylic esters to alcohols, reduction of carboxylic acids and esters to alkanes, complete reduction of epoxides, reduction of nitro compounds to amines, reduction of nitro compounds to hydroxylamines, reduction of nitroso compounds and hydroxylamines to amines, reduction of oximes to primary amines or aziridines, reduction of azides to primary amines, reduction of nitrogen compounds, and reduction of sulfonyl halides and sulfonic acids to thiols), removal of oxygen from the substrate (e.g., reduction of amine oxides and azoxy compounds, reduction of sulfoxides and sulfones, reduction of hydroperoxides and peroxides, and reduction of aliphatic nitro compounds to oximes or nitriles), reductions that include cleavage (e.g., de-alkylation of amines and amides, reduction of azo, azoxy, and hydrazo compounds to amines, and reduction of disulfides to thiols), reductive couplic reactions (e.g., bimolecular reduction of aldehydes and ketones to 1,2-diols, bimolecular reduction of aldehydes or ketones to alkenes, acyloin ester condensation, reduction of nitro to azoxy compounds, and reduction of nitro to azo compounds), and reductions in which an organic substrate is both oxidized and reduced (e.g., the Cannizzaro reaction, the Tishchenko reaction, the Pummerer rearrangement, and the Willgerodt reaction).

### IV. Selection and Screening

Selection and/or screening for reaction products with desired activities (such as catalytic activity, binding affinity, or a particular effect in an activity assay) may be performed using methodologies known and used in the art. For example, affinity selections may be performed according to the principles used in library-based selection methods such as phage display, polysome display, and mRNA-fusion protein displayed peptides. Selection for catalytic activity may be performed by affinity selections on transition-state analog affinity columns (Baca et al. (1997) PROC. NATL. ACAD. SCI. USA 94(19): 10063-8) or by function-based selection schemes (Pedersen et al. (1998) PROC. NATL. ACAD. SCI. USA 95(18): 10523-8). Since minute quantities of DNA (˜10⁻²⁰ mol) can be amplified by PCR (Kramer et al. (1999) CURRENT PROTOCOLS IN MOLECULAR BIOLOGY (ed. Ausubel, F. M.) 15.1-15.3, Wiley), these selections can be conducted on a scale ten or more orders of magnitude less than that required for reaction analysis by current methods, making a truly broad search both economical and efficient.

### (i) Selection for Binding to Target Molecule

The templates and reaction products can be selected (or screened) for binding to a target molecule. In this context, selection or partitioning means any process whereby a library member bound to a target molecule is separated from library members not bound to target molecules. Selection can be accomplished by various methods known in the art.

The templates of the present invention contain a built-in function for direct selection and amplification. In most applications, binding to a target molecule preferably is selective, such that the template and the resulting reaction product bind preferentially with a specific target molecule, perhaps preventing or inducing a specific biological effect. Ultimately, a binding molecule identified using the present invention may be useful as a therapeutic and/or diagnostic agent. Once the selection is complete, the selected templates optionally can be amplified and sequenced. The selected reaction products, if present in sufficient quantity, can be separated from the templates, purified (e.g., by HPLC, column chromatography, or other chromatographic method), and further characterized.

### (ii) Target Molecules

Binding assays provide a rapid means for isolating and identifying reaction products that bind to, for example, a surface (such as metal, plastic, composite, glass, ceramics, rubber, skin, or tissue); a polymer; a catalyst; or a target biomolecule such as a nucleic acid, a protein (including enzymes, receptors, antibodies, and glycoproteins), a signal molecule (such as cAMP, inositol triphosphate, peptides, or prostaglandins), a carbohydrate, or a lipid. Binding assays can be advantageously combined with activity assays for the effect of a reaction product on a function of a target molecule. Particular examples of targets include kinases, phosphatases, proteases, receptors, ion channels, oxidases and reductases, catabolic and anabolic enzymes, pumps, and electron transport proteins.

The selection strategy can be carried out to allow selection against almost any target. Importantly, the selection strategy does not require any detailed structural information about the target molecule or about the molecules in the libraries. The entire process is driven by the binding affinity involved in the specific recognition and binding of the molecules in the library to a given target. Examples of various selection procedures are described below.

The libraries of the present invention can contain molecules that could potentially bind to any known or unknown target. The binding region of a target molecule could include a catalytic site of an enzyme, a binding pocket on a receptor (for example, a G-protein coupled receptor), a protein surface area involved in a protein-protein or protein-nucleic acid interaction (preferably a hot-spot region), or a specific site on DNA (such as the major groove). The natural function of the target could be stimulated (agonized), reduced (antagonized), unaffected, or completely changed by the binding of the reaction product. This will depend on the precise binding mode and the particular binding site the reaction product occupies on the target.

Functional sites (such as protein-protein interaction or catalytic sites) on proteins often are more prone to bind molecules than are other more neutral surface areas on a protein. In addition, these functional sites normally contain a smaller region that seems to be primarily responsible for the binding energy: the so-called “hot-spot regions” (Wells, et al. (1993) RECENT PROG. HORMONE RES. 48: 253-262). This phenomenon facilitates selection for molecules affecting the biological function of a certain target.

The linkage between the template molecule and reaction product allows rapid identification of binding molecules using various selection strategies. This invention broadly permits identifying binding molecules for any known target molecule. In addition, novel unknown targets can be discovered by isolating binding molecules against unknown antigens (epitopes) and using these binding molecules for identification and validation. In another preferred embodiment, the target molecule is designed to mimic a transition state of a chemical reaction; one or more reaction products resulting from the selection may stabilize the transition state and catalyze the chemical reaction.

(iii) Binding Assays

The template-directed synthesis of the invention permits selection procedures analogous to other display methods such as phage display (Smith (1985) SCIENCE 228: 1315-1317). Phage display selection has been used successfully on peptides (Wells et al. (1992) CURR. OP. STRUCT. BIOL. 2: 597-604), proteins (Marks et al. (1992) J. BIOL. CHEM. 267: 16007-16010) and antibodies (Winter et al. (1994) ANNU. REV. IMMUNOL. 12: 433-455). Similar selection procedures also are exploited for other types of display systems such as ribosome display Mattheakis et al. (1994) PROC. NATL. ACAD. SCI. 91: 9022-9026) and mRNA display (Roberts, et al. (1997) PROC. NATL. ACAD. SCI. 94:12297-302). The libraries of the present invention, however, allow direct selection of target-specific molecules without requiring traditional ribosome-mediated translation. The present invention also allows the display of small molecules which have not previously been synthesized directly from a nucleic acid template.

Selection of binding molecules from a library can be performed in any format to identify optimal binding molecules. Binding selections typically involve immobilizing the desired target molecule, adding a library of potential binders, and removing non-binders by washing. When the molecules showing low affinity for an immobilized target are washed away, the molecules with a stronger affinity generally remain attached to the target. The enriched population remaining bound to the target after stringent washing is preferably eluted with, for example, acid, chaotropic salts, heat, competitive elution with a known ligand or by proteolytic release of the target and/or of template molecules. The eluted templates are suitable for PCR, leading to many orders of amplification, whereby essentially each selected template becomes available at a greatly increased copy number for cloning, sequencing, and/or further enrichment or diversification.

In a binding assay, when the concentration of ligand is much less than that of the target (as it would be during the selection of a DNA-templated library), the fraction of ligand bound to target is determined by the effective concentration of the target protein. The fraction of ligand bound to target is a sigmoidal function of the concentration of target, with the midpoint (50% bound) at [target]=K_{d} of the ligand-target complex. This relationship indicates that the stringency of a specific selection—the minimum ligand affinity required to remain bound to the target during the selection—is determined by the target concentration. Therefore, selection stringency is controllable by varying the effective concentration of target.

The target molecule (peptide, protein, DNA or other antigen) can be immobilized on a solid support, for example, a container wall, a wall of a microtiter plate well. The library preferably is dissolved in aqueous binding buffer in one pot and equilibrated in the presence of immobilized target molecule. Non-binders are washed away with buffer. Those molecules that may be binding to the target molecule through their attached DNA templates rather than through their synthetic moieties can be eliminated by washing the bound library with unfunctionalized templates lacking PCR primer binding sites. Remaining bound library members then can be eluted, for example, by denaturation.

Alternatively, the target molecule can be immobilized on beads, particularly if there is doubt that the target molecule will adsorb sufficiently to a container wall, as may be the case for an unfolded target eluted from an SDS-PAGE gel. The derivatized beads can then be used to separate high-affinity library members from nonbinders by simply sedimenting the beads in a benchtop centrifuge. Alternatively, the beads can be used to make an affinity column. In such cases, the library is passed through the column one or more times to permit binding. The column then is washed to remove nonbinding library members. Magnetic beads are essentially a variant on the above; the target is attached to magnetic beads which are then used in the selection. There are many reactive matrices available for immobilizing the target molecule, including matrices bearing —NH₂ groups or —SH groups. The target molecule can be immobilized by conjugation with NHS ester or maleimide groups covalently linked to Sepharose beads and the integrity of known properties of the target molecule can be verified. Activated beads are available with attachment sites for —NH₂ or —COOH groups (which can be used for coupling). Alternatively, the target molecule is blotted onto nitrocellulose or PVDF. When using a blotting strategy, the blot should be blocked (e.g., with BSA or similar protein) after immobilization of the target to prevent nonspecific binding of library members to the blot.

Library members that bind a target molecule can be released by denaturation, acid, or chaotropic salts. Alternatively, elution conditions can be more specific to reduce background or to select for a desired specificity. Elution can be accomplished using proteolysis to cleave a linker between the target molecule and the immobilizing surface or between the reaction product and the template. Also, elution can be accomplished by competition with a known competitive ligand for the target molecule. Alternatively, a PCR reaction can be performed directly in the presence of the washed target molecules at the end of the selection procedure. Thus, the binding molecules need not be elutable from the target to be selectable since only the template is needed for further amplification or cloning, not the reaction product itself. Indeed, some target molecules bind the most avid ligands so tightly that elution would be difficult.

To select for a molecule that binds a protein expressible on a cell surface, such as an ion channel or a transmembrane receptor, the cells themselves can be used as the selection agent. The library preferably is first exposed to cells not expressing the target molecule on their surfaces to remove library members that bind specifically or non specifically to other cell surface epitopes. Alternatively, cells lacking the target molecule are present in large excess in the selection process and separable (by fluorescence-activated cell sorting (FACS), for example) from cells bearing the target molecule. In either method, cells bearing the target molecule then are used to isolate library members bearing the target molecule (e.g., by sedimenting the cells or by FACS sorting). For example, a recombinant DNA encoding the target molecule can be introduced into a cell line; library members that bind the transformed cells but not the untransformed cells are enriched for target molecule binders. This approach is also called subtraction selection and has successfully been used for phage display on antibody libraries (Hoogenboom et al. (1998) IMMUNOTECH 4: 1-20). A selection procedure can also involve selection for binding to cell surface receptors that are internalized so that the receptor together with the selected binding molecule passes into the cytoplasm, nucleus, or other cellular compartment, such as the Golgi or lysosomes. Depending on the dissociation rate constant for specific selected binding molecules, these molecules may localize primarily within the intracellular compartments. Internalized library members can be distinguished from molecules attached to the cell surface by washing the cells, preferably with a denaturant. More preferably, standard subcellular fractionation techniques are used to isolate the selected library members in a desired subcellular compartment.

An alternative selection protocol also includes a known, weak ligand affixed to each member of the library. The known ligand guides the selection by interacting with a defined part of the target molecule and focuses the selection on molecules that bind to the same region, providing a cooperative effect. This can be particularly useful for increasing the affinity of a ligand with a desired biological function but with too low a potency.

Other methods for selection or partitioning are also available for use with the present invention. These include, for example: immunoprecipitation (direct or indirect) where the target molecule is captured together with library members; mobility shift assays in agarose or polyacrylamide gels, where the selected library members migrate with the target molecule in a gel; cesium chloride gradient centrifugation to isolate the target molecule with library members; mass spectroscopy to identify target molecules labeled with library members. In general, any method where the library member/target molecule complex can be separated from library members not bound to the target is useful.

The selection process is well suited for optimizations, where the selection steps are made in series, starting with the selection of binding molecules and ending with an optimized binding molecule. The procedures in each step can be automated using various robotic systems. Thus, the invention permits supplying a suitable library and target molecule to a fully automatic system which finally generates an optimized binding molecule. Under ideal conditions, this process should run without any requirement for external work outside the robotic system during the entire procedure.

The selection methods of the present invention can be combined with secondary selection or screening to identify reaction products capable of modifying target molecule function upon binding. Thus, the methods described herein can be employed to isolate or produce binding molecules that bind to and modify the function of any protein or nucleic acid. For example, nucleic acid-templated chemistry can be used to identify, isolate, or produce binding molecules (1) affecting catalytic activity of target enzymes by inhibiting catalysis or modifying substrate binding; (2) affecting the functionality of protein receptors, by inhibiting binding to receptors or by modifying the specificity of binding to receptors; (3) affecting the formation of protein multimers by disrupting the quaternary structure of protein subunits; or (4) modifying transport properties of a protein by disrupting transport of small molecules or ions.

Functional assays can be included in the selection process. For example, after selecting for binding activity, selected library members can be directly tested for a desired functional effect, such as an effect on cell signaling. This can, for example, be performed via FACS methodologies.

The binding molecules of the invention can be selected for other properties in addition to binding. For example, to select for stability of binding interactions in a desired working environment. If stability in the presence of a certain protease is desired, that protease can be part of the buffer medium used during selection. Similarly, the selection can be performed in serum or cell extracts or in any type of medium, aqueous or organic. Conditions that disrupt or degrade the template should however be avoided to allow subsequent amplification.

### (iv) Other Selections

Selections for other desired properties, such as catalytic or other functional activities, can also be performed. Generally, the selection should be designed such that library members with the desired activity are isolatable on that basis from other library members. For example, library members can be screened for the ability to fold or otherwise significantly change conformation in the presence of a target molecule, such as a metal ion, or under particular pH or salinity conditions. The folded library members can be isolated by performing non-denaturing gel electrophoresis under the conditions of interest. The folded library members migrate to a different position in the gel and can subsequently be extracted from the gel and isolated.

Similarly, reaction products that fluoresce in the presence of specific ligands may be selected by FACS based sorting of translated polymers linked through their DNA templates to beads. Those beads that fluoresce in the presence, but not in the absence, of the target ligand are isolated and characterized. Useful beads with a homogenous population of nucleic acid-templates on any bead can be prepared using the split-pool synthesis technique on the bead, such that each bead is exposed to only a single nucleotide sequence. Alternatively, a different anti-template (each complementary to only a single, different template) can by synthesized on beads using a split-pool technique, and then can anneal to capture a solution-phase library.

Biotin-terminated biopolymers can be selected for the actual catalysis of bond-breaking reactions by passing these biopolymers over a resin linked through a substrate to avidin. Those biopolymers that catalyze substrate cleavage self-elute from a column charged with this resin. Similarly, biotin-terminated biopolymers can be selected for the catalysis of bond-forming reactions. One substrate is linked to resin and the second substrate is linked to avidin. Biopolymers that catalyze bond formation between the substrates are selected by their ability to react the substrates together, resulting in attachment of the biopolymer to the resin.

Library members can also be selected for their catalytic effects on synthesis of a polymer to which the template is or becomes attached. For example, the library member may influence the selection of monomer units to be polymerized as well as how the polymerization reaction takes place (e.g., stereochemistry, tacticity, activity). The synthesized polymers can be selected for specific properties, such as, molecular weight, density, hydrophobicity, tacticity, stereoselectivity, using standard techniques, such as, electrophoresis, gel filtration, centrifugal sedimentation, or partitioning into solvents of different hydrophobicities. The attached template that directed the synthesis of the polymer can then be identified.

Library members that catalyze virtually any reaction causing bond formation between two substrate molecules or resulting in bond breakage into two product molecules can be selected using the schemes proposed herein. To select for bond forming catalysts (for example, hetero Diels-Alder, Heck coupling, aldol reaction, or olefin metathesis catalysts), library members are covalently linked to one substrate through their 5′ amino or thiol termini. The other substrate of the reaction is synthesized as a derivative linked to biotin. When dilute solutions of library-substrate conjugate are combined with the substrate-biotin conjugate, those library members that catalyze bond formation cause the biotin group to become covalently attached to themselves. Active bond forming catalysts can then be separated from inactive library members by capturing the former with immobilized streptavidin and washing away inactive library members

In an analogous manner, library members that catalyze bond cleavage reactions such as retro-aldol reactions, amide hydrolysis, elimination reactions, or olefin dihydroxylation followed by periodate cleavage can be selected. In this case, library members are covalently linked to biotinylated substrates such that the bond breakage reaction causes the disconnection of the biotin moiety from the library members. Upon incubation under reaction conditions, active catalysts, but not inactive library members, induce the loss of their biotin groups. Streptavidin-linked beads can then be used to capture inactive polymers, while active catalysts are able to be eluted from the beads. Related bond formation and bond cleavage selections have been used successfully in catalytic RNA and DNA evolution (Jaschke et al. (2000) CURR. OPIN. CHEM. BIOL. 4: 257-62) Although these selections do not explicitly select for multiple turnover catalysis, RNAs and DNAs selected in this manner have in general proven to be multiple turnover catalysts when separated from their substrate moieties (Jäschke et al. (2000) CURR. OPIN. CHEM. BIOL. 4: 257-62; Jaeger et al. (1999) PROC. NATL. ACAD. SCI. USA 96: 14712-7; Bartel et al. (1993) SCIENCE 261: 1411-8; Sen et al. (1998) CURR. OPIN. CHEM. BIOL. 2: 680-7).

In addition to simply evolving active catalysts, the in vitro selections described above are used to evolve non-natural polymer libraries in powerful directions difficult to achieve using other catalyst discovery approaches. Substrate specificity among catalysts can be selected by selecting for active catalysts in the presence of the desired substrate and then selecting for inactive catalysts in the presence of one or more undesired substrates. If the desired and undesired substrates differ by their configuration at one or more stereocenters, enantioselective or diastereoselective catalysts can emerge from rounds of selection. Similarly, metal selectivity can be evolved by selecting for active catalysts in the presence of desired metals and selecting for inactive catalysts in the presence of undesired metals. Conversely, catalysts with broad substrate tolerance can be evolved by varying substrate structures between successive rounds of selection.

Importantly, in vitro selections can also select for specificity in addition to binding affinity. Library screening methods for binding specificity typically require duplicating the entire screen for each target or non-target of interest. In contrast, selections for specificity can be performed in a single experiment by selecting for target binding as well as for the inability to bind one or more non-targets. Thus, the library can be pre-depleted by removing library members that bind to a non-target. Alternatively, or in addition, selection for binding to the target molecule can be performed in the presence of an excess of one or more non-targets. To maximize specificity, the non-target can be a homologous molecule. If the target molecule is a protein, appropriate non-target proteins include, for example, a generally promiscuous protein such as an albumin. If the binding assay is designed to target only a specific portion of a target molecule, the non-target can be a variation on the molecule in which that portion has been changed or removed.

### (vi) Amplification and Sequencing

Once all rounds of selection are complete, the templates which are, or formerly were, associated with the selected reaction product preferably are amplified using any suitable technique to facilitate sequencing or other subsequent manipulation of the templates. Natural oligonucleotides can be amplified by any state of the art method. These methods include, for example, polymerase chain reaction (PCR); nucleic acid sequence-based amplification (see, for example, Compton (1991) NATURE 350: 91-92), amplified anti-sense RNA (see, for example, van Gelder et al. (1988) PROC. NATL. ACAD. SCI. USA 85: 77652-77656); self-sustained sequence replication systems (Gnatelli et al. (1990) PROC. NATL. ACAD. SCI. USA 87: 1874-1878); polymerase-independent amplification (see, for example, Schmidt et al. (1997) NUCLEIC ACIDS RES. 25: 4797-4802, and in vivo amplification of plasmids carrying cloned DNA fragments. Descriptions of PCR methods are found, for example, in Saiki et al. (1985) SCIENCE 230: 1350-1354; Scharf et al. (1986) SCIENCE 233: 1076-1078; and in U.S. Pat. No. 4,683,202. Ligase-mediated amplification methods such as Ligase Chain Reaction (LCR) may also be used. In general, any means allowing faithful, efficient amplification of selected nucleic acid sequences can be employed in the method of the present invention. It is preferable, although not necessary, that the proportionate representations of the sequences after amplification reflect the relative proportions of sequences in the mixture before amplification.

For non-natural nucleotides the choices of efficient amplification procedures are fewer. As non-natural nucleotides can be incorporated by certain enzymes including polymerases it will be possible to perform manual polymerase chain reaction by adding the polymerase during each extension cycle.

For oligonucleotides containing nucleotide analogs, fewer methods for amplification exist. One may use non-enzyme mediated amplification schemes (Schmidt et al. (1997) NUCLEIC ACIDS RES. 25: 4797-4802). For backbone-modified oligonucleotides such as PNA and LNA, this amplification method may be used. Alternatively, standard PCR can be used to amplify a DNA from a PNA or LNA oligonucleotide template. Before or during amplification the templates or complementing templates may be mutagenized or recombined in order to create an evolved library for the next round of selection or screening.

(vii) Sequence Determination and Template Evolution

Sequencing can be done by a standard dideoxy chain termination method, or by chemical sequencing, for example, using the Maxam-Gilbert sequencing procedure. Alternatively, the sequence of the template (or, if a long template is used, the variable portion(s) thereof) can be determined by hybridization to a chip. For example, a single-stranded template molecule associated with a detectable moiety such as a fluorescent moiety is exposed to a chip bearing a large number of clonal populations of single-stranded nucleic acids or nucleic acid analogs of known sequence, each clonal population being present at a particular addressable location on the chip. The template sequences are permitted to anneal to the chip sequences. The position of the detectable moieties on the chip then is determined. Based upon the location of the detectable moiety and the immobilized sequence at that location, the sequence of the template can be determined. It is contemplated that large numbers of such oligonucleotides can be immobilized in an array on a chip or other solid support.

Libraries can be evolved by introducing mutations at the DNA level, for example, using error-prone PCR (Cadwell et al. (1992) PCR METHODS APPL. 2: 28) or by subjecting the DNA to in vitro homologous recombination (Stemmer (1994) PROC. NATL. ACAD. SCI. USA 91: 10747; Stemmer (1994) NATURE 370: 389) or by cassette mutagenesis.

### (a) Error-Prone PCR

Random point mutagenesis is performed by conducting the PCR amplification step under error-prone PCR (Cadwell et al. (1992) PCR METHODS APPLIC. 2: 28-33) conditions. Because the genetic code of these molecules are written to assign related codons to related chemical groups, similar to the way that the natural protein genetic code is constructed, random point mutations in the templates encoding selected molecules will diversify progeny towards chemically related analogs. Because error-prone PCR is inherently less efficient than normal PCR, error-prone PCR diversification is preferably conducted with only natural dATP, dTTP, dCTP, and dGTP and using primers that lack chemical handles or biotin groups.

### (b) Recombination

Libraries may be diversified using recombination. For example, templates to be recombined may have a structure in which codons are separated by five-base non-palindromic restriction endonuclease cleavage sites such as those cleaved by AvaII (G/GWCC, W=A or T), Sau96I (G/GNCC, N=A, G, T, or C), DdeI (C/TNAG), or HinFI (G/ANTC). Following selections, templates encoding desired molecules are enzymatically digested with these commercially available restriction enzymes. The digested fragments then are recombined into intact templates with T4 DNA ligase. Because the restriction sites separating codons are nonpalindromic, template fragments can only reassemble to form intact recombined templates. DNA-templated translation of recombined templates provides recombined small molecules. In this way, functional groups between synthetic small molecules with desired activities are recombined in a manner analogous to the recombination of amino acid residues between proteins in Nature. It is well appreciated that recombination explores the sequence space of a molecule much more efficiently than point mutagenesis alone (Minshull et al. (1999) CURR. OPIN. CHEM. BIOL. 3: 284-90; Bogarad et al. (1999) PROC. NATL. ACAD. SCI. USA 96: 2591-5; Stemmer NATURE 370: 389-391).

A preferred method of diversifying library members is through nonhomologous random recombination, as described, for example, in WO 02/074978; US Patent Application Publication No. 2003-0027180-A1; and Bittker et al. (2002) NATURE BIOTECH. 20(10): 1024-9.

### (c) Random Cassette Mutagenesis

Random cassette mutagenesis is useful to create a diversified library from a fixed starting sequence. Thus, such a method can be used, for example, after a library has been subjected to selection and one or more library members have been isolated and sequenced. Generally, a library of oligonucleotides with variations on the starting sequence is generated by traditional chemical synthesis, error-prone PCR, or other methods. For example, a library of oligonucleotides can be generated in which, for each nucleotide position in a codon, the nucleotide has a 90% probability of being identical to the starting sequence at that position, and a 10% probability of being different. The oligonucleotides can be complete templates when synthesized, or can be fragments that are subsequently ligated with other oligonucleotides to form a diverse library of templates.

The following examples contain important additional information, exemplification and guidance that can be adapted to the practice of this invention in its various embodiments and equivalents thereof. Practice of the invention will be more fully understood from these following examples, which are presented herein for illustrative purpose only, and should not be construed as limiting in anyway.

### EXAMPLE

### DNA-Templated Synthesis in Organic Solvents

### (i) General Experimental Materials and Methods

Acetonitrile, N,N′-dimethylformamide (DMF), Dichloromethane (DCM) and tetrahydrofuran (THF) solvents used for DNA-templated chemistry were purchased from J. T. Baker (Cylcetainer™) and passed through a neutral alumina column prior to use. Chemicals were purchased from Sigma-Aldrich unless otherwise noted.

Oligonucleotides were synthesized on a Perseptive Biosystems Expedite 8090 DNA synthesizer using standard phosphoramidite protocols and purified using preparative scale reverse-phase HPLC. Reagents for automated solid-phase oligonucleotide synthesis were purchased from Glen Research. Functionalized DNA oligonucleotides were purified by analytical scale reverse-phase HPLC. Concentrations of purified oligonucleotides in solution were determined based on their absorbance at 260 nm measured on a Hewlett-Packard 8453 UV-visible spectrophotometer (Agilent Technologies). Oligonucleotides stained with ethidium bromide were visualized and quantitated by UV transillumination and densitometry using an Eagle Eye II densitometer (Stratagene).

Denaturing PAGE analysis was performed using 15% polyacrylamide gel (TBE-urea). Yield calculations assumed that species in denaturing gels stain with comparable intensity per nucleotide. MALDI-TOF mass spectrometry was performed on an Applied Biosystems Voyager-DE Pro Biospectrometry Workstation and processed with Voyager Data Explorer software. A mixture of nine parts hydroxypicolinic acid (HPA, 50 mg/mL in 50% MeCN/H₂O) and one part ammonium citrate (50 mg/mL in H₂O) was used as the matrix in all experiments. Karl Fischer water content analysis was performed using an Aquastar Karl Fischer coulometric titrator model C400.

### (ii) Preparation and Characterization of Functionalized DNA Oligonucleotides

Amino-Terminated and Biotinylated DNA Oligonucleotides. 5′ amino-modifier 5 (Glen Research) was used to prepare 5′ amino-modified oligonucleotides. 3′ amino-modifier C7 and 3′ amino-modifier PTC3 CPG (Glen Research) was used to prepare 3′ amino-modified oligonucleotides. Biotin TEG CPG (Glen Research) was used to prepare 3′ biotin-linked oligonucleotides used for MALDI analysis.

DNA Oligonucleotides Linked to Other Small Molecules. In a typical small-molecule conjugation reaction, a small-molecule carboxylic acid was pre-activated as an N-hydroxysuccinimide (NHS) ester by combining 0.9 M solutions of the carboxylic acid, NHS and 1,3-dicyclohexyl carbodiimide (DCC) in a 1:1:1 stoichiometry in dimethylformamide (DMF) and letting the reaction proceed at 25° C. for 1 hour. The dicyclohexylurea precipitate was pelleted by microcentrifugation and the supernatant was used directly in a DNA conjugation reaction. For those substrates that were commercially available as activated carboxylic acid derivatives, a 10 mg/mL stock solution in DMF was prepared and added directly to the conjugation reaction. A typical DNA conjugation reaction consisted of 10 nmol of amino-terminated oligonucleotide in 100 μL of 0.2 M phosphate buffer at pH 8.0, to which 45 μL of the NHS ester solution was added. After 2 hours at 25° C., the reaction mixture was directly loaded onto a Nap-5 size exclusion column (Amersham Biosciences) to remove organic solvent, salts, and excess small molecules, and was further purified by analytical scale reverse-phase HPLC (8-30% MeCN/0.1 M TEAA gradient). The desired oligonucleotide products were characterized by MALDI-TOF mass spectrometry. The structures of the small molecule-linked oligonucleotides are shown in Table 2. See Table 3 for MALDI characterization of each substrate-linked oligonucleotide.

TABLE 2
DNA-Linked Small Molecule Structures

| Structure | Reagents |
|---|---|
| 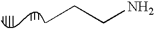 | 1a-g, 2a, 6a, 7a |
| 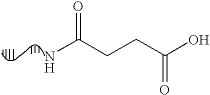 | 2b, 7c |
| 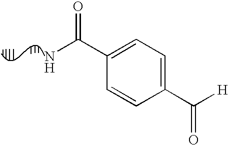 | 2c, 7d |
| 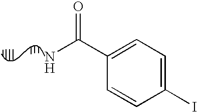 | 2d, 6c |
| 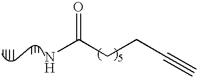 | 2e, 6d |
| 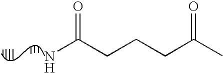 | 3a-d, 6b |
| 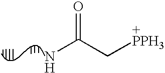 | 4a-d, 6e |
| 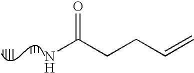 | 5a-d, 7b |

DNA Sequences Used to Carry out DTS in Organic Solvents. The DNA sequences used to carry out DTS in organic solvents were as follows (listed in the 5′ to 3′ direction:

(SEQ ID NO: 1)

E1 (10-mer): AATTCGTACC
(exact mass: 2986.58)

(SEQ ID NO: 2)

E10 (10-mer): TCCCGAGTCG
(exact mass: 3003.56)

(SEQ ID NO: 3)

mis (10-mer): GCTAGAGCCT
(exact mass: 3027.57)

(SEQ ID NO: 4)

Ω5: TCCCGAGTCGGTACC
(exact mass: 4527.82)

(SEQ ID NO: 5)

E1 (20-mer): TCCCGAGTCGAATTCGTACC
(exact mass: 6051.09)

(SEQ ID NO: 6)

E10 (20-mer): AAGGTGGTATTCCCGAGTCG
(exact mass: 6171.01)

(SEQ ID NO: 7)

mis (20-mer): TGACGACACTATATCAAGCC
(exact mass: 6068.11)

(SEQ ID NO: 8)

T30: GGTACGAATTCGACTCGGGAATACCACCTT
(exact mass: 9187.63)

(SEQ ID NO: 9)

M20: TCCCGAGTCGAATTCGTACC
(exact mass: 6051.09)

(SEQ ID NO: 10)

M10: GGTACGAATT
(exact mass: 3066.58)

TABLE 3
MALDI mass spectroscopic characterization
of DNA-linked small-molecule reagents

| | | | Small Molecule | | |
|---|---|---|---|---|---|
| Reagent | | | (see above for | Expected | Observed |
| No. | Reagent Type | Resin | structure) | Mass | Mass |
| 1a | E1(10-mer) | 3′ amino C7 | none (free amine) | 3195.08 | 3195 ± 5 |
| 1b | E10(10-mer) | 3′ amino PTC3 | none (free amine) | 3140.58 | 3141 ± 5 |
| 1c | mis(10-mer) | 3′ amino PTC3 | none (free amine) | 3164.59 | 3170 ± 5 |
| 1d | Ω5 | 3′ amino C7 | none (free amine) | 4736.90 | 4743 ± 7 |
| 1e | E1(20-mer) | 3′ amino C7 | none (free amine) | 6260.17 | 6266 ± 9 |
| 1f | E10(20-mer) | 3′ amino C7 | none (free amine) | 6380.09 | 6388 ± 9 |
| 1g | mis(20-mer) | 3′ amino PTC3 | none (free amine) | 6205.13 | 6210 ± 9 |
| 2a | T30 | 5′ amino | none (free amine) | 9354.66 | 9359 ± 12 |
| | | modifier | | | |
| 2b | T30 | 5′ amino | succinic acid | 9454.68 | 9454 ± 12 |
| | | modifier | | | |
| 2c | T30 | 5′ amino | p-formyl benzoic | 9486.69 | 9485 ± 12 |
| | | modifier | acid | | |
| 2d | T30 | 5′ amino | iodo-benzoic acid | 9584.59 | 9582 ± 12 |
| | | modifier | | | |
| 2e | T30 | 5′ amino | nonynoic acid | 9490.75 | 9503 ± 12 |
| | | modifier | | | |
| 3a | E1(10-mer) | 3′ amino C7 | 5-oxohexanoic acid | 3307.71 | 3310 ± 5 |
| 3b | E10(10-mer) | 3′ amino PTC3 | 5-oxohexanoic acid | 3253.21 | 3256 ± 5 |
| 3c | mis(10-mer) | 3′ amino PTC3 | 5-oxohexanoic acid | 3276.64 | 3276 ± 5 |
| 3d | Ω5 | 3′ amino PTC3 | 5-oxohexanoic acid | 4776.90 | 4780 ± 7 |
| 4a | E1(10-mer) | 3′ amino C7 | phosphorane | 3501.20 | 3503 ± 5 |
| 4b | E10(10-mer) | 3′ amino PTC3 | phosphorane | 3492.71 | 3490 ± 5 |
| 4c | mis(10-mer) | 3′ amino PTC3 | phosphorane | 3468.70 | 3470 ± 5 |
| 4d | Ω5 | 3′ amino PTC3 | phosphorane | 4968.95 | 4975 ± 7 |
| 5a | E1(20-mer) | 3′ amino PTC3 | pentenoic acid | 6270.16 | 6275 ± 9 |
| 5b | E10(20-mer) | 3′ amino PTC3 | pentenoic acid | 6390.08 | 6387 ± 9 |
| 5c | mis(20-mer) | 3′ amino PTC3 | pentenoic acid | 6287.18 | 6286 ± 9 |
| 5d | Ω5 | 3′ amino PTC3 | pentenoic acid | 4746.89 | 4746 ± 7 |

(iii) DNA-Templated Chemistry in Aqueous Solvent

Amine Acylation in Aqueous Solvent (FIG. 1C, lanes 1-4). Succinic acid-linked 30-mer 2b (8 pmol) and amino-terminated reagent 1a-d (12 pmol) were incubated in 3 μL 70 mM NaCl in H₂O. Aqueous 100 mM N-[3-morpholinopropane]-sulfonic acid (MOPS), NaCl (1 m)M, pH 7.0 in the presence of 40 mM EDC and 25 mM N-hydroxysulfosuccinimide (sNHS) was added to initiate reaction in a total volume of 300 μL. Reactions were allowed to proceed at 25° C. for 12 hours before being precipitated by the addition of 0.1 volumes of 20 μg/mL glycogen in 3 m NaOAc, pH 5.0 and 2.5 volumes of EtOH.

Amine acylation in aqueous solution (100 mM MOPS, pH 7.0, 1 M NaCl, 40 mM EDC, 25 mM sNHS) was found to proceed in 81, 58, and 84% yield for the E1, E10, and Ω5 architectures, respectively, with no significant product formation from mismatched reagent and template oligonucleotides (FIG. 1C, lanes 1-4).

Reductive Amination in Aqueous Solvent (FIG. 2A lanes 1-4). Aldehyde-linked 30-mer 2c (8 pmol) and amino-terminated reagent 1a-d (12 pmol) were incubated in 3 μL 70 mM NaCl in H₂O, N-[2-morpholinoethane]sulfonic acid (MES) buffer (100 mM), NaBr (1 M), pH 6.0 (177 μL) was added. 100 μL of NaBH₃CN (240 mM in MES buffer) and 20 μL of acetic acid (200 mM in H₂O) were added to initiate reaction. Reactions were allowed to proceed at 25° C. for 12 hours before being quenched with the addition of 20 μL of 1 M dithiothreitol (DTT) in H₂O. Reactions were then precipitated by the addition of 0.1 volumes of 20 μg/mL glycogen in 3 M NaOAc, pH 5.0 and 2.5 volumes of EtOH.

As shown in FIG. 2A, lanes 1-4, reductive amination produced products from E1, E10, and 25 architectures; however no significant products were observed from mismatched reagent and template oligonucleotides.

Wittig Olefination in Aqueous Solvent (FIG. 2A, lanes 5-8). Aldehyde-linked 30-mer 2c (8 pmol) and phosphorane-linked reagent 4a-d (12 pmol) were incubated in 3 μL 70 mM NaCl in H₂O. Aqueous MOPS buffer (100 mM), NaCl (1 M), pH 7.0 was added with 10 mM TEA for a total reaction volume of 300 μL. Reactions were allowed to proceed at 25° C. for 12 hours before being precipitated by the addition of 0.1 volumes of 20 μg/mL glycogen in 3 M NaOAc, pH 5.0 and 2.5 volumes of EtOH.

As shown in FIG. 2A, lanes 5-8, Wittig olefination produced products from E1, E10, and Ω5 architectures; however no significant products were observed from mismatched reagent and template oligonucleotides.

Ene-Yne Oxidative Coupling in Aqueous Solvent (FIG. 2A, lanes 9-12). Alkyne-linked 30-mer 2e (8 pmol) and pentene-linked reagent 5a-d (12 pmol) were incubated in 3 μL 70 mM NaCl in H₂O. MOPS buffer (100 mM), NaCl (1 M), pH 7.0 (285 μL) and 25 mM Na₂PdCl₄ in H₂O (12 μL) were added. Reactions were allowed to proceed at 37° C. for 4 hours and precipitated by the addition of EtOH as described above. Pellets were resuspended in 300 μL 20 mm DTT in 100 mM MES and heated to 95° C. for 10 minutes. Heating resulted in the formation of a precipitate that was removed by centrifugal filtration (Ultrafree-MC centrifugal filtration devices from Millipore). Following filtration, samples were precipitated by the addition of 0.1 volumes of 20 μg/mL glycogen in 3 M NaOAc, pH 5.0 and 2.5 volumes of EtOH.

As shown in FIG. 2A, lanes 9-12, Ene-Yne oxidative coupling produced products from the E1, E10, and Ω5 architectures; however no significant products were observed from mismatched reagent and template oligonucleotides.

Heck Coupling in Aqueous Solvent (FIG. 5, lanes 5-8). Aryl iodide-linked 30-mer 2d (8 pmol) and pentene-linked reagent 5a-d (12 μmol) were incubated in 3 μL 70 mm NaCl in H₂O. MES buffer (100 mM), NaBr (1 M), pH 6.0 (267 μL) was added. A suspension of 10 mM Pd₂(dba)₃-CHCl₃ in H₂O was added for a total reaction volume of 300 μL. Reactions were allowed to proceed at 25° C. for 16 hours before being precipitated by the addition of 0.1 volumes of 20 μg/mL glycogen in 3 M NaOAc, pH 5.0 and 2.5 volumes of EtOH. Pellets were resuspended in 300 μL 20 mM DTT in 100 mM MES and heated to 95° C. for 10 minutes, subjected to centrifugal filtration to remove precipitate and precipitated by the addition of EtOH. No significant products were observed by gel electrophoresis (see FIG. 5, lanes 5-8).

Enamine Aldol Chemistry in Aqueous Solvent (FIG. 5, lanes 1-4). Aldehyde-linked 30-mer 2c (8 pmol) and ketone-linked reagent 3a-d (12 pmol) were incubated in 3 μL 70 mm NH₄Cl in H₂O. MOPS buffer (100 mm), NaCl (1 M), pH 7.0 with 50 mM pyrrolidine in H₂O was added for a total volume of 300 μL. Reactions were allowed to proceed at 25° C. for 16 h before being precipitated by the addition of 0.1 volumes of 20 μg/mL glycogen in 3 M NaOAc, pH 5.0 and 2.5 volumes of EtOH. No significant products were observed by gel electrophoresis (FIG. 5, lanes 1-4).

### (iv) DNA-Templated Chemistry in 95% Organic Solvent

Amine Acylation in Acetonitrile (95% MeCN. 5% H₂O, FIG. 1C, lanes 5-8). Succinic acid-linked 30-mer 2b (8 pmol) and amino-terminated reagent 1a-d (12 pmol) were incubated in 3 μL 70 mM NaCl in H₂O. 185 μL of MeCN and 100 μL of a solution of 120 mM 1-(3-dimethylaminopropyl)-3-ethyl carbodiimide (EDC) and 75 mM N-hydroxysuccinimide (NHS) was added. Reaction volume was adjusted to 300 μL by the addition of 12 μL of H₂O. Reactions were allowed to proceed at 25° C. for 12 hours before being quenched with the addition of 20 μL of 1 M methyl amine in acetonitrile. Reactions were then precipitated by the addition of 0.1 volumes of 20 μg/mL glycogen in 3 M NaOAc, pH 5.0 and 2.5 volumes of EtOH. Under these conditions, the E1, E10, and Ω5 architectures all generated amide products efficiently (88, 82, and 70% yield, respectively), as characterized by denaturing PAGE and MALDI mass spectrometry (FIG. 1C, lanes 5-7; Table 1); however, mismatched reagent and template oligonucleotides showed no significant product formation (see FIG. 1C, lane 8).

Amine Acylation in Acetonitrile Without Pre-Hybridization (95% MeCN 5% H, FIG. 1c, lanes 9-12). Succinic acid-linked 30-mer 2b (8 pmol) was incubated in 2 μL 100 mm NaCl in H₂O. 185 μL of MeCN and 100 μL of a solution of 120 mM 1-(3-dimethylaminopropyl)-3-ethyl carbodiimide (EDC) and 75 mM N-hydroxysuccinimide (NHS) was added. Amino terminated reagent 1a-d was added directly (12 pmol) and the reaction volume was adjusted to 300 μL by the addition of 12 μL of H₂O. Reactions were allowed to proceed at 25° C. for 12 hours before being quenched with the addition of 20 μL of 1 M methyl amine in MECN. Reactions were then precipitated by the addition of 0.1 volumes of 20 μg/mL glycogen in 3 M NaOAc, pH 5.0 and 2.5 volumes of EtOH.

As shown in FIG. 1C, lanes 9-12, no significant product formation was observed without pre-hybridization.

Reductive Amination in Dimethylformamide (95% DMF, 5% H₂O; FIG. 2B, lanes 1-4). Aldehyde-linked 30-mer 2c (8 pmol) and amino-terminated reagent 1a-d (12 pmol) were incubated in 3 μL 70 mM NaCl in H₂O. 165 μL of DMF, 100 μL of NaBH₃CN (240 mM in DMF), and 20 μL of acetic acid (200 mM in DMF) were added. Reaction volume was adjusted to 300 μL by the addition of 12 μL of H₂O. Reactions were allowed to proceed at 25° C. for 12 hours before being quenched with the addition of 20 μL of 1 m dithiothreitol (DTT) in DMF. Reactions were then precipitated by the addition of 0.1 volumes of 20 μg/mL glycogen in 3 M NaOAc, pH 5.0 and 2.5 volumes of EtOH.

In 95% N,N′-dimethylformamide (DMF), the E1, E10, and Ω5 architectures generated reductive amination products in 59, 5, and 45% yield, respectively (FIG. 2B, lanes 1-3); however, no significant product formation was observed from mismatched template and reagent oligonucleotides (FIG. 2B, lane 4).

Wittig Olefination in Acetonitrile (95% MeCN, 5% H₂O; FIG. 2B lanes 5-8). Aldehyde-linked 30-mer 2c (8 pmol) and phosphorane-linked reagent 4a-d (12 pmol) were incubated in 3 μL 70 mM NaCl in H₂O. 285 μL of acetonitrile were added directly and reaction was initiated with the addition of 12 μL of NaOH (250 mM in H₂O). Reactions were allowed to proceed at 25° C. for 12 hours and quenched with 20 μL of 200 mM benzaldehyde in acetonitrile before being precipitated by the addition of 0.1 volumes of 20 μg/mL glycogen in 3 M NaOAc, pH 5.0 and 2.5 volumes of EtOH.

Wittig chemistry was found to proceed efficiently in 95% acetonitrile with product yields exceeding 90% in the E1, E10, and Ω5 architectures (FIG. 2B, lanes 5-7); however no significant product formation was observed from mismatched reagent and template oligonucleotides (FIG. 2B, lane 8).

Ene-Yne Oxidative Coupling in Acetonitrile (95% MeCN, 5% H₂O: FIG. 2B, lanes 9-12). Alkyne-linked 30-mer 2e (8 pmol) and pentene-linked reagent 5a-d (12 pmol) were incubated in 3 μL 70 mM NaCl in H₂O. MeCN (285 μL) and 25 mM Na₂PdCl₄ in H₂O (12 μL) was added for a final reaction volume of 300 μL with 95% MeCN. Reactions were allowed to proceed at 37° C. for 4 hours before being precipitated by the addition of 0.1 volumes of 20 μg/mL glycogen in 3 M NaOAc, pH 5.0 and 2.5 volumes of EtOH. Pellets were resuspended in 300 μL 20 mm dithiothreitol (DTT) in 100 mM N-[2-morpholinoethane]-sulfonic acid (MES) and heated to 95° C. for 10 minutes. Heating resulted in the formation of a precipitate that was removed by filtration. Following filtration, samples were precipitated by the addition of 0.1 volumes of 20 μg/mL glycogen in 3 M NaOAc, pH 5.0 and 2.5 volumes of EtOH. In 95% acetonitrile, enone products were generated in 71, 60, and 63% yield in the E1, E10, and Ω5 architectures, respectively (FIG. 2B, lanes 9-11), with no significant product formation observed from mismatched reagent and template oligonucleotides (FIG. 2B, lane 12).

Heck Coupling in Tetrahydrofuran (95% THE: FIG. 3, lanes 5-8). Aryl iodide-linked 30-mer 2d (8 pmol) and pentene-linked reagent 5a-d (12 pmol) were incubated in 3 μL 70 mM NaCl in H₂O. THF (255 μL) and 10 mM Pd₂(dba)₃-CHCl₃ in THF (30 μL) were added. 12 μL of H₂O were added for a total reaction volume of 300 μL. Reactions were allowed to proceed at 25° C. for 16 hours before being precipitated by the addition of 0.1 volumes of 20 μg/mL glycogen in 3 M NaQAc, pH 5.0 and 2.5 volumes of EtOH. Pellets were resuspended in 300 μL 20 mM DTT in 100 mM MES and heated to 95° C. for 10 minutes. Heating resulted in the formation of a precipitate that was removed by filtration. Samples were precipitated by the addition of 0.1 volumes of 20 μg/mL glycogen in 3 M NaOAc, pH 5.0 and 2.5 volumes of EtOH. Heck coupling was found to proceed sequence-specifically in yields of 91%, 85%, and 80% in the E1, E10, and Ω5 architectures, respectively, in 95% tetrahydrofuran (THF) (FIG. 3, lanes 5-7); however, no significant product formation was observed from mismatched reagent and template oligonucleotides (FIG. 3, lane 8).

Enamine Aldol Chemistry in Acetonitrile (95% MeCN 5% H20: FIG. 3, lanes 1-4). Aldehyde-linked 30-mer 2c (8 pmol) and ketone-linked reagent 3a-d (12 pmol) were incubated in 3 μL 70 mm NH₄Cl in H₂O. 255 μL of MeCN and 30 μL of 500 mm pyrrolidine in MeCN (30 μL) were added. The total reaction volume was adjusted to 300 μL with the addition of 12 μL of H₂O. Reactions were allowed to proceed at 25° C. for 16 hours before being precipitated by the addition of 0.1 volumes of 20 μg/mL glycogen in 3 M NaOAc, pH 5.0 and 2.5 volumes of EtOH.

In 95% acetonitrile, the E1, E10, and Ω5 architectures generated aldol condensation products in 88%, 79%, and 82% yield, respectively, with no significant product formation from mismatched reagent and template oligonucleotides (FIG. 3, lanes 1-4, Table 1).

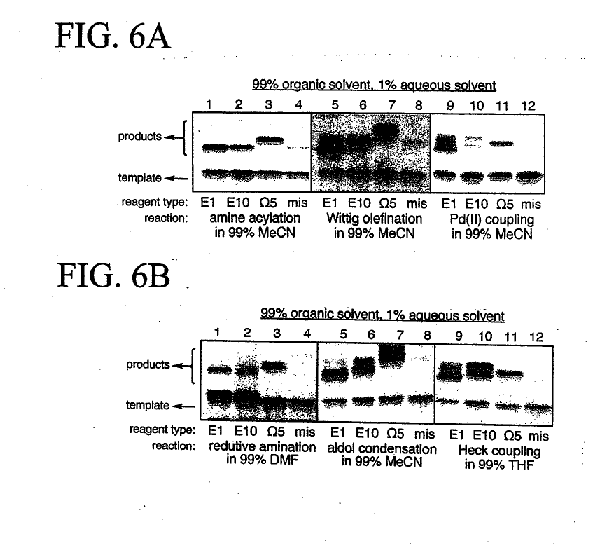

### (v) DNA-Templated Chemistry in 99% Organic Solvent (see FIG. 6)

Amine Acylation in Acetonitrile (99% MeCN, 1% H₂O). Succinic acid-linked 30-mer 2b (8 pmol) and amino-terminated reagent 1a-d (12 pmol) were incubated in 3 μL 70 mm NaCl in H₂O. 197 μL of MeCN and 100 μL of a solution of 120 mM 1-(3-dimethylaminopropyl)-3-ethyl carbodiimide (EDC) and 75 mM N-hydroxy-succinimide (NHS) was added. Reactions were allowed to proceed at 25° C. for 12 hours before being quenched with the addition of 20 μL of 1 M methyl amine in acetonitrile. Reactions were then precipitated by the addition of 0.1 volumes of 20 μg/mL glycogen in 3 M NaOAc, pH 5.0 and 2.5 volumes of EtOH.

As shown in FIG. 6A, lanes 1-4, amine acylation produced products for the E1, E10, and Ω5 architectures; however no significant products were observed from mismatched reagent and template oligonucleotides.

Reductive Amination in Dimethylformamide (99% DMF. 1% H₂O). Aldehyde-linked 30-mer 2c (8 pmol) and amino-terminated reagent 1a-d (12 pmol) were incubated in 3 μL 70 mM NaCl in H₂O. 177 μL of DMF, 100 μL of NaBH₃CN (240 mM in DMF), and 20 μL of acetic acid (200 mM in DMF) was added to initiate reaction and 100 μL of a solution of 240 mM NaBH₃CN in DMF was added. Reactions were allowed to proceed at 25° C. for 12 hours before being quenched with the addition of 20 μL of 1 M dithiothreitol (DTT) in DMF. Reactions were then precipitated by the addition of 0.1 volumes of 20 μg/mL glycogen in 3 M NaOAc, pH 5.0 and 2.5 volumes of EtOH.

As shown in FIG. 6B, lanes 1-4, reductive amination produced products for the E1, E10, and Ω5 architectures; however no significant products were observed from mismatched reagent and template oligonucleotides.

Wittig Olefination in Acetonitrile (99% MeCN, 1% H₂O). Aldehyde-linked 30-mer 2c (8 pmol) and phosphorane-linked reagent 4a-d (12 pmol) were incubated in 3 μL 70 mM NaCl in H₂O. 297 μL of 100 mM TEA in acetonitrile were added directly to initiate reaction. Reactions were allowed to proceed at 25° C. for 12 hours and quenched with 20 μL of 200 mm benzaldehyde in acetonitrile before being precipitated by the addition of 0.1 volumes of 20 μg/mL glycogen in 3 M NaOAc, pH 5.0 and 2.5 volumes of EtOH.

As shown in FIG. 6A, lanes 5-8, Wittig olefination produced products for the E1, E10, and Ω5 architectures; however no significant products were observed from mismatched reagent and template oligonucleotides.

Ene-Yne Oxidative Coupling in Acetonitrile (99% MeCN, 1% H₂O). Alkyne-linked 30-mer 2e (8 pmol) and pentene-linked reagent 5a-d (12 pmol) were incubated in 3 μL 70 mm NaCl in H₂O. Pre-hybridized oligos were lyophilized to dryness and MeCN (297 μL) and 3 μL Na₂PdCl₄ (100 mM in H₂O) was added for a final reaction volume of 300 μL with 99% MeCN and 1% H₂O. Reactions were allowed to proceed at 25° C. for 6 hours before being precipitated by the addition of 0.1 volumes of 20 μg/mL glycogen in 3 M NaQAc, pH 5.0 and 2.5 volumes of EtOH. Pellets were resuspended in 300 μL 20 mM dithiothreitol (DTT) in 100 mM N-[2-morpholinoethane]sulfonic acid (MES) and heated to 95° C. for 10 minutes. Heating resulted in the formation of a precipitate that was removed by filtration. Following filtration, samples were precipitated by the addition of 0.1 volumes of 20 μg/mL glycogen in 3 M NaOAc, pH 5.0 and 2.5 volumes of EtOH.

As shown in FIG. 6A, lanes 9-12, Ene-Yne oxidative coupling produced products for the E1, E10, and Ω5 architectures; however no significant products were observed for mismatched reagent and template oligonucleotides.

Heck Coupling in Tetrahydrofuran (99% THF, 9% H₂O). Aryl iodide-linked 30-mer 2d (8 pmol) and pentene-linked reagent 5a-d (12 pmol) were incubated in 3 μL 70 mm NaCl in H₂O. THF (267 μL) and 10 mM Pd₂(dba)₃-CHCl₃ in THF (30 μL) were added. Reactions were allowed to proceed at 25° C. for 16 hours before being precipitated by the addition of 0.1 volumes of 20 μg/mL glycogen in 3 M NaOAc, pH 5.0 and 2.5 volumes of EtOH. Pellets were resuspended in 300 μL 20 mM DTT in 100 mM MES and heated to 95° C. for 10 minutes. Heating resulted in the formation of a precipitate that was removed by filtration. Samples were precipitated by the addition of 0.1 volumes of 20 μg/mL glycogen in 3 M NaOAc, pH 5.0 and 2.5 volumes of EtOH.

Significant Heck coupling product formation was observed when reactions were carried out in 99% THF (see FIG. 6B, lanes 9-11), with no significant product formation from mismatched reagent and template oligonucleotides (FIG. 6B, lane 12).

Enamine Aldol Chemistry in Acetonitrile (99% MeCN, 1% H₂O). Aldehyde-linked 30-mer 2c (8 pmol) and ketone-linked reagent 3a-d (12 pmol) were incubated in 3 μL 70 mM NaCl in H₂O. 267 μL of MeCN and 30 μL of 500 mM pyrrolidine in MeCN (30 μL) were added. Reactions were allowed to proceed at 25° C. for 16 hours before being precipitated by the addition of 0.1 volumes of 20 μg/mL glycogen in 3 M NaOAc, pH 5.0 and 2.5 volumes of EtOH.

As shown in FIG. 6B, lanes 5-8, aldol condensation produced products for the E1, E10, and Ω5 architectures; however no significant products were observed for mismatched reagent and template oligonucleotides.

### (vi) DNA-Templated Chemistry in 99.9% Organic Solvent

To study DNA-templated chemistry in dry organic solvents pre-hybridized DNA-linked reactants were frozen and lyophilized to dryness before being dissolved in anhydrous organic solvents. A Karl Fischer test was performed to ascertain the final H₂O content of the dry DTS reactions in water-miscible solvents. Testing the anhydrous solvents directly revealed the following H₂O content: MeCN, 65 ppm; DMF, 202 ppm, and THF, 200 ppm. As a representative model case, succinic acid-linked 30-mer 2b (8 pmol) and amino-terminated reagent (12 pmol) were incubated in 3 μL 70 mM NaCl in H₂O. The pre-hybridized oligos were frozen and lyophilized to dryness before being re-suspended in 300 μL of anhydrous organic solvent and analyzed by Karl Fischer to reveal the following: MeCN 10mer reagent, 356 ppm; MeCN 15mer reagent, 377 ppm; MeCN 20mer reagent, 329 ppm; DMF 10mer reagent, 533 ppm; DMF 15mer reagent, 542 ppm; DMF 20mer reagent, 599 ppm; THF 10mer reagent, 416 ppm; THF 15mer reagent, 448 ppm; and THF 20mer reagent, 468 ppm. The observed range of 300-600 ppm corresponds to a final organic solvent concentration of >99.9%.

Wittig Olefination in Acetonitrile (>99.9% MeCN, <0.1% H₂O: FIG. 4, lanes 1-4). Aldehyde-linked 30-mer 2c (8 pmol) and phosphorane-linked reagent 4a-d (12 pmol) were incubated in 3 μL 70 mM NaCl in H₂O. The pre-hybridized oligos were frozen and lyophilized to dryness before the reaction was initiated. 300 μL of 100 mM TEA in acetonitrile were added directly. Reactions were allowed to proceed at 25° C. for 12 hours and quenched with 20 μL of 200 mM benzaldehyde in acetonitrile before being precipitated by the addition of 0.1 volumes of 20 μg/mL glycogen in 3 M NaOAc, pH 5.0 and 2.5 volumes of EtOH.

Sequence-specific Wittig product formation under these conditions was observed (FIG. 4, lanes 1-3), albeit at lower yields (10-56%) than in 95% organic solvents (see FIG. 2B, lanes 5-7). As in 95% solvent, no significant product formation was observed when reagent and template oligonucleotides were mismatched (FIG. 4, lane 4).

Enamine Aldol Chemistry in Acetonitrile (>99.9% MeCN, <0.1% H₂O; FIG. 4, lanes 5-8) Aldehyde-linked 30-mer 2c (8 pmol) and ketone-linked reagent 3a-d (12 pmol) were incubated in 3 μL 70 mM NH₄Cl in H₂O. Pre-hybridized oligos were lyophilized to dryness. 270 μL of MeCN and 30 μL of 500 mM pyrrolidine in MeCN (30 μL) were added. Reactions were allowed to proceed at 25° C. for 16 h before being precipitated by the addition of 0.1 volumes of 20 μg/mL glycogen in 3 M NaOAc, pH 5.0 and 2.5 volumes of EtOH.

Sequence-specific aldol product formation under these conditions was observed (FIG. 4, lanes 5-7), albeit at lower yields (10-56%) than in 95% organic solvents (FIG. 3, lanes 1-3). As in 95% solvent, no significant product formation was observed from mismatched reagent and template oligonucleotides (FIG. 4, lane 8).

Amine Acylation in Dichloromethane (>99.9% DCM, <0.1% H₂O; FIG. 4, lanes 9-12). Succinic acid-linked 30-mer 2b (8 pmol) and amino-terminated reagent 1a-d (12 pmol) were incubated in 3 μL 70 mM NaCl in H₂O. Pre-hybridized oligos were frozen and lyophilized to dryness. CH₂Cl₂ (200 μL) was added. Reaction was initiated with the addition of 100 μL of a solution of 120 mM 1-(3-dimethylaminopropyl)-3-ethyl carbodiimide (EDC) and 75 mM N-hydroxysuccinimide (NHS) in CH₂Cl₂. Reactions were allowed to proceed at 25° C. for 12 hours before being quenched with the addition of 20 μL of 1 M methyl amine in CH₂Cl₂. Reactions were precipitated by the addition of 0.1 volumes of 20 μg/mL glycogen in 3 M NaOAc, pH 5.0 and 2.5 volumes of EtOH.

Amine acylation in >99.9% dichloromethane (DCM) containing 40 mM of water-insoluble dicyclohexyl carbodiimide (DCC) and 25 mM NHS proceeded only in low efficiencies (21% for the E1 architecture and <10% yield for the E10 and Ω5 architectures, (FIG. 4, lanes 9-12, Table 1), although sequence-specificity was retained.

(vii) Titrating Water in the Aldol Reaction

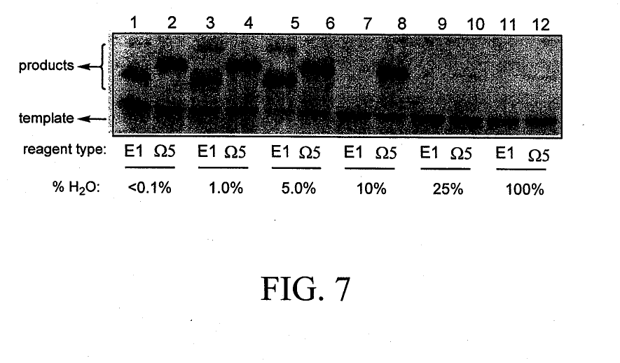

Pyrrolidine-mediated aldol chemistry was performed in acetonitrile containing ˜0.1%, 1.0%, 5.0%, 25%, 50% H₂O as well as in pure aqueous conditions. All reactions were performed in the E1 and Ω5 architectures. In all cases below, aldehyde-linked 30-mer 2c (8 pmol) and ketone-linked 10-mer 3a-d (12 pmol) were incubated in 3 μL 70 mm NH₄Cl in H₂O, See FIG. 7.

˜0.1% H₂O. Reactions were performed as described above, in section (vi) of this Example.

1.0% H₂O. Reactions were performed as described above, in section (v) of this Example.

5.0% H₂O. Reactions were performed as described above, in section (iv) of this Example.

25.0% H₂O. MeCN (195 μL) and 500 mM pyrrolidine in MeCN (30 μL) and H₂O (200 mM NaCl) (72 μL) were added for a total reaction volume of 300 μL.

50.0% H₂O. MeCN (120 μL) and 500 mm pyrrolidine in MeCN (30 μL) and H₂O (200 mM NaCl) (147 μL) were added for a total reaction volume of 300 μL.

100% H₂O. Aqueous reactions were performed as described above, in section (iii) of this Example.

As shown in FIG. 7, the aldol reaction produced products for the E1 and Ω5 architectures in organic solvent/water mixtures containing up to 5% water. At 10% water content, the Ω5 architecture produced product but the E1 architecture did not. Above 10% water content, neither architecture appeared to produce product via the aldol reaction.

(viii) DNA-Templated Chemistry in Organic Solvents with Alkyl Ammonium Salts

It was initially hypothesized that a short (10-30 bp) DNA duplex formed in aqueous solution and transferred to an organic solvent containing low concentrations (μM) of quaternary ammonium salts would retain its double-stranded structure, as shown in FIG. 8.

Experiments showed that DNA-templated chemistry could indeed take place efficiently and sequence-specifically in organic solvents in the presence of alkyl ammonium salts. DNA-templated chemistries were investigated in four distinct contexts: i) in a simple end-of-helix architecture with juxtaposed reactants (E1), ii) in a long-distance end-of-helix architecture with ten intervening nucleotides between hybridized reactants (E10), iii) in the “omega” architecture with a five-base loop (Ω5), and iv) with reactants linked to noncomplementary (mismatched) oligonucleotides.

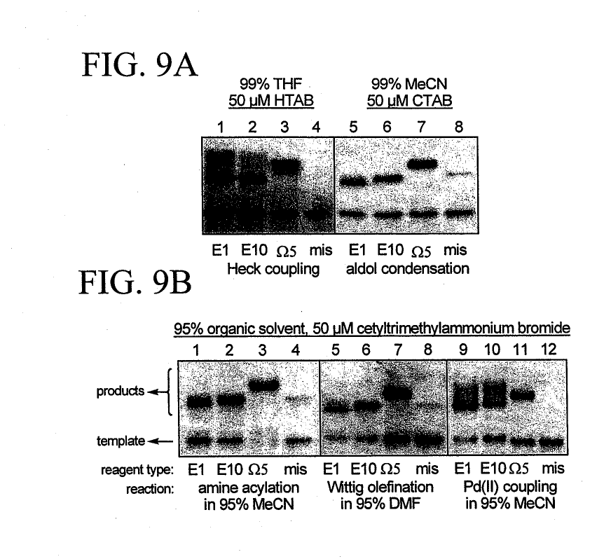

Five representative examples of DTS in organic solvents in the presence of cetyltrimethyl ammonium bromide (CTAB) or hexadecyltrimethyl ammonium bromide (HTAB) are shown in FIG. 9. Reactions were performed exactly as described in Section (iv) and Section (v) of this Example, with the addition of 50 μM CTAB or HTAB in the final (300 μL) reaction volume. Denaturing PAGE analysis of amine acylation, Wittig olefination, Pd(II) mediated oxidative ene-yne coupling, Heck coupling, and aldol condensation chemistry are depicted in FIG. 9.

For amine acylation under prehybridized conditions, in a final solvent composition of 95% acetonitrile with 5% water and 50 μM cetyltrimethylammonium bromide (CTAB) the E1, E10, and Ω5 architectures generated amide products in 58, 63, and 82% yield, respectively, with no product yield for mismatched reagent and template (see FIG. 9B, lanes 1-4). Similarly, Heck coupling (FIG. 9A, lanes 1-4), aldol condensation (FIG. 9A, lanes 5-8), Wittig olefination (FIG. 9B, lanes 5-8), and Ene-Yne oxidative coupling (FIG. 9B, lanes 9-12) all showed product yield for the E1, E10, and Ω5 architectures, with no significant product yield for mismatched reagent and template.

### (ix) MALDI-TOF Analysis of DNA-Templated Reaction Products

DNA-templated chemistry performed in organic solvents was also characterized by MALDI-TOF mass spectrometry. Two oligonucleotides were used for these experiments: a 10-base oligonucleotide conjugated to a small-molecule at the 5′ end and a 20-base oligonucleotide (such that the 10-mer was complementary to a 10-base region of the 20-mer) with a 5′ biotin group and a small-molecule attached to the 3′ end through a base-labile sulfone linker. The 10-base oligonucleotide was synthesized with a 5′ amine using the 5′ amino-modifier 5 phosphoramidite and conjugated to small-molecule reagents using the procedures described above. The 20-base oligonucleotide was synthesized with a 3′ amine and a 5′ biotin using the 3′ amino-modifier C7 CPG and the 5′ biotin phosphoramidite. The oligonucleotide was conjugated to a substrate via a cleavable linker in a two-step procedure. In the first step, 5-10 nmol of oligonucleotide in 100 μL of 0.2 M phosphate pH 8.0 was combined with 10 μL of a 0.1 M solution of bis[2-(succinimidyloxycarbonyloxy)-ethyl]sulfone (BSOCOES, Pierce) in DMF and mixed by vortexing for 90 seconds.

Immediately after vortexing, 9 μL of a 0.2 M solution of hexanediamine dihydrochloride salt in H₂O was added and the solution was agitated at 25° C. for 1 hour. The solution was then passed through a Nap-5 gel filtration column (Amersham) and the hexanediamine-linked oligonucleotide was purified by reverse-phase HPLC. The purified oligonucleotide was then conjugated to the appropriate carboxylic acid using the procedures described in section (ii) of this Example, Preparation and Characterization of Functionalized DNA Oligonucleotides. Substrate-linked 20-base (M20) and 10-base (M10) oligonucleotides used in these experiments were characterized by MALDI-TOF mass spectroscopy as shown in Table 4.

TABLE 4
MALDI-TOF characterization of DNA-linked
small-molecule substrates

| | | | Expected | Observed |
|---|---|---|---|---|
| ID | Oligo Type | Small-molecule | Mass | Mass |
| 6a | M10 | 5′ amino 5 modifier | 3233.62 | 3230 ± 5 |
| 6b | M10 | 4-oxopentanoic acid | 3347.68 | 3348 ± 5 |
| 6c | M10 | p-iodobenzoic acid | 3463.54 | 3467 ± 5 |
| 6d | M10 | phospho-ylide | 3537.72 | 3538 ± 5 |
| 6e | M10 | nonynoic acid | 3369.70 | 3370 ± 5 |
| 7a | M20 | free hexamine | 6915.38 | 6918 ± 9 |
| 7b | M20 | pentenoic acid | 6997.42 | 6998 ± 9 |
| 7c | M20 | succinic acid | 7015.40 | 7021 ± 9 |
| 7d | M20 | p-formyl benzoic | 7048.41 | 7051 ± 9 |
| | | acid | | |

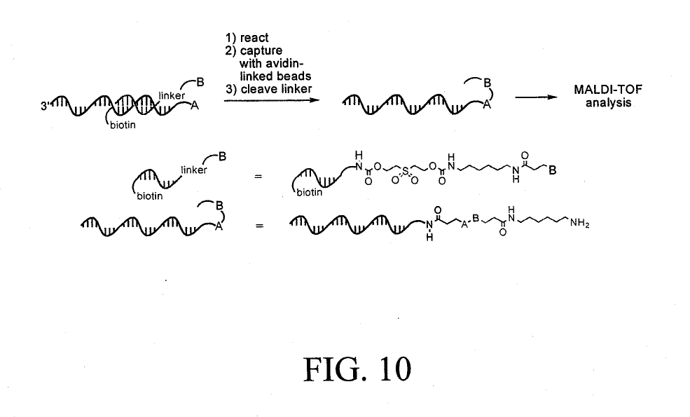

To obtain a product sample suitable for MALDI-TOF analysis, 50 pmol of substrate-linked 20-mer was combined with 75 pmol of substrate-linked 10-mer were pre-incubated and exposed to reaction conditions as described above. Following the final EtOH precipitation step, pellets were resuspended in 300 μL 10 mM Tris, 0.1 M NaCl, 1 mM EDTA, pH 8.2 and added to an aliquot of streptavidin magnetic particles (Roche) representing 80 pmol of biotinylated oligonucleotide binding capacity. After 15 minutes, the supernatant was removed and the particles were washed twice with 150 μL Milli-Q H₂O. The particles were then resuspended in 0.1 M CAPS, pH 11 and agitated for 15 min to effect sulfone linker cleavage (FIG. 10). The supernatant was subjected to centrifugal filtration and subsequent EtOH precipitation. The pellet was resuspended in 0.1 M TEAA, desalted using a Zip-Tip cartridge (Millipore), and subjected to MALDI-TOF analysis, yielding the results in Table 1.

### INCORPORATION BY REFERENCE

The entire disclosure of each of the publications and patent documents referred to herein is incorporated by reference in its entirety for all purposes to the same extent as if each individual publication or patent document were so individually denoted.

### EQUIVALENTS

The invention may be embodied in other specific forms without departing form the spirit or essential characteristics thereof. The foregoing embodiments are therefore to be considered in all respects illustrative rather than limiting on the invention described herein. Scope of the invention is thus indicated by the appended claims rather than by the foregoing description, and all changes that come within the meaning and range of equivalency of the claims are intended to be embraced therein.

What is claimed is:

1. A method of performing nucleic acid templated synthesis to produce a reaction product, the method comprising:
(a) providing a solution comprising (i) a template comprising a first reactive unit associated with a first oligonucleotide defining a first codon sequence, and (ii) a transfer unit comprising a second reactive unit associated with a second oligonucleotide defining a first anti-codon sequence complementary to the first codon sequence of the template;
(b) annealing the first codon and first anti-codon sequences to bring the first reactive unit and the second reactive unit into reactive proximity; and
(c) after step (b), inducing a reaction between the first and second reactive units in a solution comprising an organic solvent to produce a reaction product.

2. The method of claim 1, comprising the additional step of adding a solution containing an organic solvent to the product of step (b).

3. A method of performing nucleic acid templated synthesis to produce a reaction product, the method comprising:
(a) providing in a solution comprising an organic solvent (i) a template comprising a first reactive unit associated with a first oligonucleotide defining a first codon sequence, and (ii) a transfer unit comprising a second reactive unit associated with a second oligonucleotide defining a first anti-codon sequence complementary to the first codon sequence of the template;
(b) annealing the first codon and first anti-codon sequences to bring the first reactive unit and the second reactive unit into reactive proximity; and
(c) inducing a reaction between the first and second reactive units to produce a reaction product.

4. The method of claim 3, wherein all of the steps (a), (b) and (c) are performed in a single solution comprising an organic solvent.

5. The method of claim 1, wherein in the template, the first reactive unit is associated with the first oligonucleotide at a location adjacent to an end of the first oligonucleotide.

6. The method of claim 1, wherein in the template, the first reactive unit is associated with the first oligonucleotide at a location at least 2 bases from an end of the first oligonucleotide.

7. The method of claim 6, wherein the first reactive unit is associated with the first oligonucleotide at a location at least 5 bases from an end of the first oligonucleotide.

8. The method of claim 7, wherein the first reactive unit is associated with the first oligonucleotide at a location at least 10 bases from an end of the first oligonucleotide.

9. The method of claim 1, wherein the template is capable of producing an omega or a single stranded loop structure when annealed to the transfer unit.

10. The method of claim 1, wherein in the template, the first reactive unit is covalently attached to the first oligonucleotide.

11. The method of claim 1, wherein at least one organic solvent is selected from CH₃CN, DMF, THF, CH₃OH, C₂H₅OH, CH₂Cl₂, CCl₄, CHCl₃, toluene, benzene, diethyl ether, glyme, hexanes, and DMSO.

12. The method of claim 1, wherein at least one organic solvent is selected from CH₃CN, DMF, THF, CH₃OH, and CHCl₃.

13. The method of claim 1, wherein the organic solvent is a solvent other than CH₂Cl₂.

14. The method of claim 1, wherein the second reactive unit is covalently attached to the second oligonucleotide.

15. The method of claim 1, wherein the template further comprises a second, different codon sequence.

16. The method of claim 1, further comprising providing a second transfer unit that anneals to the second, different codon sequence of the template.

17. The method of claim 16, wherein the first and second transfer units are provided together in step (b).

18. The method of claim 1, further comprising the additional step of selecting reaction product associated with the template.

19. The method of claim 1, wherein the reaction product is covalently attached to the template.

20. The method of claim 1, further comprising the additional step of amplifying the template.

21. The method of claim 1, further comprising the additional step of determining the sequence of the template thereby to facilitate identification of the reaction product.

22. The method of claim 1, wherein the nucleic acid templated reaction can also be performed in an aqueous medium.

23. The method of claim 1, wherein the nucleic acid templated reaction is water-incompatible.

24. The method of claim 1, wherein the nucleic acid templated reaction is a carbon-carbon bond formation reaction.

25. The method of claim 1, wherein at least one of the template and transfer unit is solublized by one or more quaternary ammonium ions.

26. The method of claim 1, wherein the reaction in organic solvent produces a smaller yield of product than the reaction in an aqueous solvent.

27. The method of claim 1, wherein the reaction in organic solvent produces a greater yield of product than the reaction in an aqueous solvent.

28. The method of claim 1, wherein the reaction occurs in a solution comprising 10% (v/v)-100% (v/v) organic solvent.

29. The method of claim 1, wherein the reaction occurs in a solution comprising 30% (v/v)-80% (v/v) organic solvent.

30. A method for identifying a compound having binding affinity to a target molecule, the method comprising:
(a) performing one or more nucleic acid-templated reactions to produce one or more compounds each covalently linked to a corresponding oligonucleotide having a nucleotide sequence informative of the synthetic history or structure of the compound, wherein at least one of the nucleic acid-templated reactions is performed in a solution comprising an organic solvent;
(b) mixing the compounds and a target molecule under conditions to permit the compounds capable of binding the target molecule to bind thereto;
(c) separating the compounds that bind to the target molecule from unbound compounds; and
(d) identifying the oligonucleotide associated with a compound that binds to the target molecule as indicative of binding affinity of the compound to the target molecule.

31. The method of claim 30, wherein at least one organic solvent is selected from the group consisting of CH₃CN, DMF, THF, CH₃OH, CH₂Cl₂ and CHCl₃.

32. The method of claim 30, wherein step (d) comprises determining the sequence of the oligonucleotide associated with a compound that binds to the target molecule.

33. The method of claim 30 further comprising, after step (c) but before step (d), the step of amplifying the oligonucleotides associated with the separated compounds.

34. The method of claim 30, wherein the nucleotide sequence encodes the synthesis of the compound associated therewith.

35. The method of claim 30, wherein the target molecule is a protein.

36. The method of claim 1, wherein the method includes one or more chemical reactions not mediated by nucleic acid templates.

37. The method of claim 1, wherein the method includes one or more chemical reactions that involve reactants not associated with oligonucleotides.

38. The method of claim 1, wherein the method includes one or more chemical reactions that involve reactants not covalently linked to oligonucleotides.

39. A library of compounds prepared by the method of claim 1.

40. A reaction product produced by the method of claim 1.


SEQUENCE LISTING (10 sequences)

SEQ ID NO: 1 - DNA, 10 bp, Artificial Sequence
  DNA sequences used to carry out DNA-templated synthesis (DTS) in organic solvents
aattcgtacc

SEQ ID NO: 2 - DNA, 10 bp, Artificial Sequence
  DNA sequences used to carry out DNA-templated synthesis (DTS) in organic solvents
tcccgagtcg

SEQ ID NO: 3 - DNA, 10 bp, Artificial Sequence
  DNA sequences used to carry out DNA-templated synthesis (DTS) in organic solvents
gctagagcct

SEQ ID NO: 4 - DNA, 15 bp, Artificial Sequence
  DNA sequences used to carry out DNA-templated synthesis (DTS) in organic solvents
tcccgagtcggtacc

SEQ ID NO: 5 - DNA, 20 bp, Artificial Sequence
  DNA sequences used to carry out DNA-templated synthesis (DTS) in organic solvents
tcccgagtcgaattcgtacc

SEQ ID NO: 6 - DNA, 20 bp, Artificial Sequence
  DNA sequences used to carry out DNA-templated synthesis (DTS) in organic solvents
aaggtggtattcccgagtcg

SEQ ID NO: 7 - DNA, 20 bp, Artificial Sequence
  DNA sequences used to carry out DNA-templated synthesis (DTS) in organic solvents
tgacgacactatatcaagcc

SEQ ID NO: 8 - DNA, 30 bp, Artificial Sequence
  DNA sequences used to carry out DNA-templated synthesis (DTS) in organic solvents
ggtacgaattcgactcgggaataccacctt

SEQ ID NO: 9 - DNA, 20 bp, Artificial Sequence
  DNA sequences used to carry out DNA-templated synthesis (DTS) in organic solvents
tcccgagtcgaattcgtacc

SEQ ID NO: 10 - DNA, 10 bp, Artificial Sequence
  DNA sequences used to carry out DNA-templated synthesis (DTS) in organic solvents
ggtacgaatt